(19) United States
(12) Patent Application Publication
(10) Pub. No.: US 2026/0283458 A1
(43) Pub. Date: Sep. 24, 2026

(54) IMAGING CAMERA, MOBILE APPLICATION, AND HEALTH MONITORING SYSTEM

(71) Applicant: Pacific Dental Services, LLC, Henderson, NV (US)

(72) Inventors: Mostafa Khairzada, Mission Viejo, CA (US); Luke De Ruyter, Anaheim, CA (US); David Baker, Henderson, NV (US); Karine Deledalle, Torrance, CA (US); Heidi Liu, Trabuco Canyon, CA (US); Brijesh Patel, Irvine, CA (US); Abdullah Kazi, Pleasonton, CA (US); Trace Stephens, Ladera Ranch, CA (US); Laila Ayubi, Irvine, CA (US)

(21) Appl. No.: 19/571,199

(22) Filed: Mar. 18, 2026

Related U.S. Application Data
(60) Provisional application No. 63/774,676, filed on Mar. 19, 2025.

(51) Int. Cl. A61B 1/24; A61B 1/00; A61B 1/04; A61B 1/05; A61B 1/06; A61B 1/12; A61B 90/00

(52) U.S. Cl. CPC A61B 1/24; A61B 1/000095; A61B 1/000096; A61B 1/0004; A61B 1/00045; A61B 1/00055; A61B 1/00105; A61B 1/043; A61B 1/053; A61B 1/0607; A61B 1/0684; A61B 1/127; A61B 2090/365

(57) ABSTRACT

A health monitoring system is disclosed that includes a handheld intraoral imaging device, a mobile computing device executing a mobile application, and a multi-layer artificial intelligence (AI) analysis framework. The handheld intraoral imaging device includes an image sensor, lens assembly, illumination sources, wireless communication module, processor, and memory, and is configured to capture intraoral image data and transmit the image data to the mobile computing device. The AI analysis framework is executed by at least one of the handheld intraoral imaging device and the mobile computing device. The AI analysis framework filters non-relevant or low-quality images, identifies anatomical regions within retained images, associates retained images with specific teeth or oral regions, and generates longitudinal condition indicators by comparing historical and current image data. The mobile application presents the longitudinal condition indicators to a user to support oral health monitoring and risk-awareness.

### CROSS-REFERENCE TO RELATED APPLICATIONS

This application claims priority to U.S. Provisional Patent Application No. 63/774,676, filed on Mar. 19, 2025, the disclosure of which is incorporated herein by reference in its entirety for all purposes.

### TECHNICAL FIELD

The disclosure relates generally to medical and dental imaging systems, and more specifically, and not by way of limitation, to intraoral imaging devices, mobile applications, and artificial intelligence-based analysis frameworks for capturing, analyzing, and longitudinally monitoring oral health conditions.

### BACKGROUND OF THE INVENTION

Some example embodiments relate to medical and dental imaging cameras. More particularly, some example embodiments relate to medical and dental imaging cameras configured for use in home monitoring and documentation systems.

Technological advancements in the field of dentistry include the intraoral camera (IOC). Intraoral cameras enable visualization of regions within the oral cavity that may otherwise be difficult to observe during routine examinations. Intraoral cameras further generate dental images that may be recorded for purposes including patient education, clinical documentation, treatment planning, and professional presentation. Intraoral cameras that capture images within the oral cavity are widely used in dental offices throughout North America.

The camera may be generally positioned at a distal end of an intraoral wand and transmits real-time video for review by a dental professional and a patient. Such visual communication tools have improved patient understanding and engagement. Intraoral cameras are commonly used chairside to display images of the oral cavity, to assist discussion of treatment options, and to store images within a patient record for later reference.

Medical imaging systems, including cameras, have also been developed for capturing and storing images of a patient's body for use in clinical observation, documentation, and assessment across a variety of medical fields, including dermatology. For example, International PCT Patent Application WO2020101380A1 describes a camera system that utilizes three-dimensional shape extraction technology to analyze surface characteristics of skin tissue for assisting in clinical evaluation.

However, many such high-quality dental and medical imaging systems are primarily available within clinical environments. Due to factors such as inconvenience, scheduling limitations, and cost, patients may delay visiting medical or dental offices, which can reduce opportunities for early observation and monitoring of developing conditions.

More recently, smartphones have been used to capture medical images for review by medical professionals. For example, Korean Patent Publication No. KR20060034557A describes using a mobile phone to capture and transmit images of the human body for remote professional evaluation. However, smartphones are not specifically designed for consistent, high-quality acquisition of dental and medical images, particularly for intraoral or specialized anatomical imaging.

Accordingly, there remains a need for a camera system suitable for home use that is capable of obtaining high-quality dental and medical images in a reliable and repeatable manner. There is also a need for an in-home camera system capable of securely transmitting images to remote locations for professional monitoring and clinical evaluation by medical and dental practitioners. Furthermore, there is a need for an in-home camera system capable of automatically tracking previously identified dental and medical condition indicators over time to support longitudinal observation, documentation, and user awareness. Some embodiments may provide for one or more of these.

### SUMMARY

The present disclosure provides a camera-based health monitoring system configured for capturing anatomical image data, processing the image data using artificial intelligence, and generating longitudinal condition indicators for user monitoring. The system supports repeated image acquisition, automated image quality control, anatomical region identification, and temporal comparison of image data to enable detection of condition changes over time.

In one embodiment, a health monitoring system includes a handheld intraoral imaging device having an image sensor, lens assembly, illumination sources, a wireless communication module, a processor, and memory, a mobile computing device executing a mobile application, and a multi-layer artificial intelligence analysis framework executed by at least one of the handheld device and the mobile computing device. The handheld device captures intraoral image data and transmits the image data to the mobile computing device. The artificial intelligence analysis framework filters non-relevant or low-quality images, identifies anatomical regions within retained images, associates retained images with specific teeth or oral regions, and generates longitudinal condition indicators by comparing historical and current image data. The mobile application presents the longitudinal condition indicators to a user.

In another embodiment, a method of monitoring oral health conditions includes capturing intraoral image data using a handheld imaging device, transmitting the image data to a mobile computing device, filtering the image data using a first artificial intelligence model, evaluating image clarity using a second artificial intelligence model, identifying anatomical regions within retained images using a third artificial intelligence model, associating the retained images with specific oral regions, comparing the retained images with historical image data to determine longitudinal condition indicators, and displaying the longitudinal condition indicators to a user through a mobile application.

### BRIEF DESCRIPTION OF THE DRAWINGS

FIG. 1 is an exploded perspective view of the dental and medical camera in accordance with the systems, devices, and methods described herein;

FIG. 2 is an advertising image illustrating the dental and medical camera in accordance with the systems, devices, and methods described herein;

FIG. 3 is a diagram illustrating a preferred heating system for minimizing fogging of the camera lens in accordance with the systems, devices, and methods described herein;

FIG. 4 is a graph illustrating a preferred heating system including 18 resistors for minimizing fogging of the camera lens as compared to 9 standard LEDs in accordance with the systems, devices, and methods described herein;

FIG. 5 is a first image illustrating placement of LEDs upon a printed circuit board around the lens of the camera in accordance with the systems, devices, and methods described herein;

FIG. 6 is a second image illustrating placement of LEDs upon a printed circuit board around the lens of the camera in accordance with the systems, devices, and methods described herein;

FIG. 7 is an image of the flexible printed circuit board shown in FIGS. 5 and 6;

FIG. 8 is an exploded perspective view of the lens assembly of the dental and medical camera in accordance with the systems, devices, and methods described herein;

FIG. 9 includes side-by-side images showing improved imaging produced by the dental and medical camera in accordance with the systems, devices, and methods described herein;

FIG. 10 includes two images of the dental and medical camera's primary circuit board illustrating how the placement of the electrical components on the PCB reduced signal interferences in accordance with the systems, devices, and methods described herein;

FIG. 11 includes screen shots of the mobile application (App) for use with the dental and medical camera in accordance with the systems, devices, and methods described herein;

FIG. 12 illustrates an example patient dashboard in accordance with the systems, devices, and methods described herein;

FIG. 13 illustrates the camera and applications in accordance with the systems, devices, and methods described herein;

FIGS. 14A-14C illustrates an example user flow in accordance with the systems, devices, and methods described herein;

FIG. 15 is a diagram illustrating a device, that may connect to an electric toothbrush and/or an intraoral camera in accordance with the systems, devices, and methods described herein;

FIG. 16 illustrates how the intraoral camera wirelessly connects to the mobile application for real-time video streaming, AI analysis, and data transmission in accordance with the systems, devices, and methods described herein;

FIG. 17 illustrates an example user interface displaying AI-detected oral pathologies ranked by severity with descriptions in accordance with the systems, devices, and methods described herein;

FIG. 18 illustrates a habit tracker within the mobile application for logging daily brushing, flossing, and tongue cleaning in accordance with the systems, devices, and methods described herein;

FIG. 19 illustrates an example user interface displaying a health score metric and a streak tracker indicating consecutive days of completed oral care activities in accordance with the systems, devices, and methods described herein;

FIG. 20 illustrates a daily check-in interface where users answer questions about their oral hygiene habits for that day in accordance with the systems, devices, and methods described herein;

FIG. 21 illustrates a welcome survey interface collecting baseline oral health information when a user first sets up a profile in accordance with the systems, devices, and methods described herein; and

FIG. 22 is a flowchart of an example method in accordance with the systems, devices, and methods described herein.

FIG. 23 illustrates an example user interface 2300 for guiding a device-connection workflow between a base unit 2302 and an imaging attachment 2304.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Unless expressly stated otherwise, specific implementation details described herein, including component selections, numeric values, materials, processing frameworks, communication protocols, model architectures, and operating parameters, are provided by way of example only and are not intended to limit the scope of the disclosed systems and methods. Other implementations may use different components, values, configurations, or techniques while achieving similar functionality.

Several aspects of example systems will now be presented with reference to various apparatus and methods. These apparatus and methods will be described in the following detailed description and illustrated in the accompanying drawings by various blocks, components, circuits, processes, algorithms, etc. (collectively referred to as “elements”). These elements may be implemented using various components, hardware, electronic hardware, computer software, or any combination thereof. Whether such elements are implemented as hardware or software depends upon the particular application and design constraints imposed on the overall system.

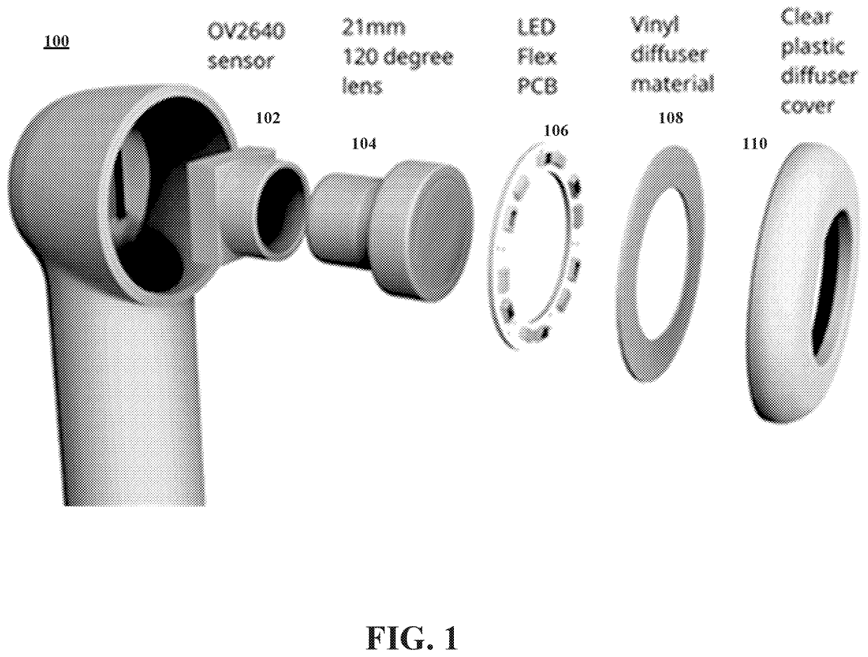

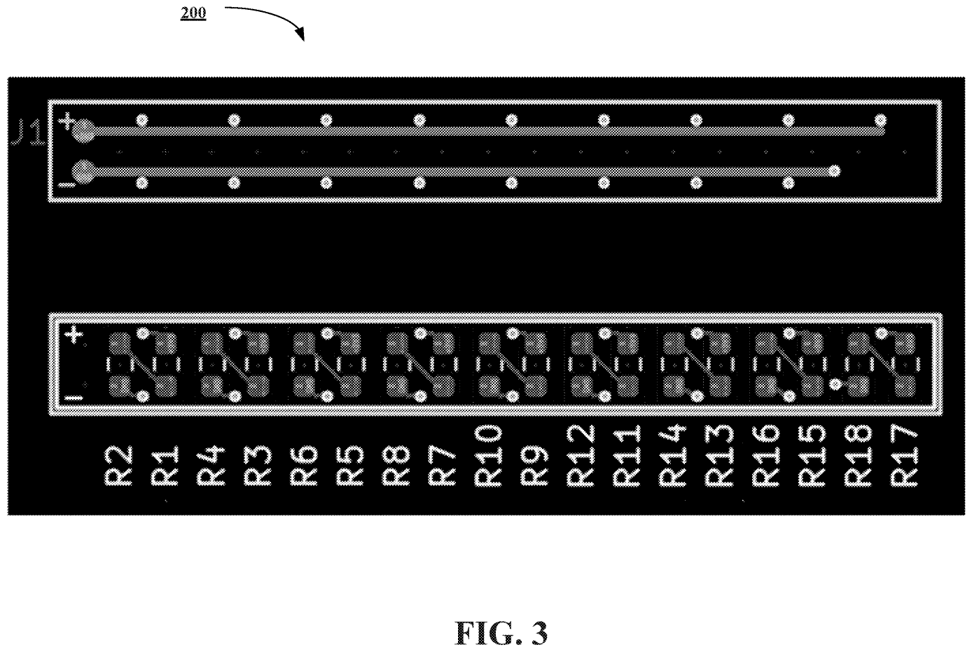

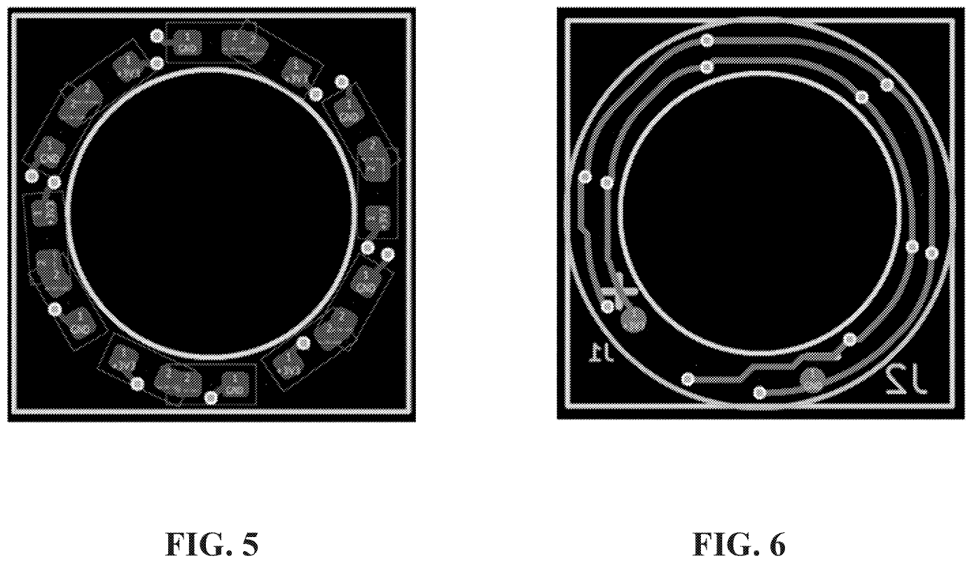

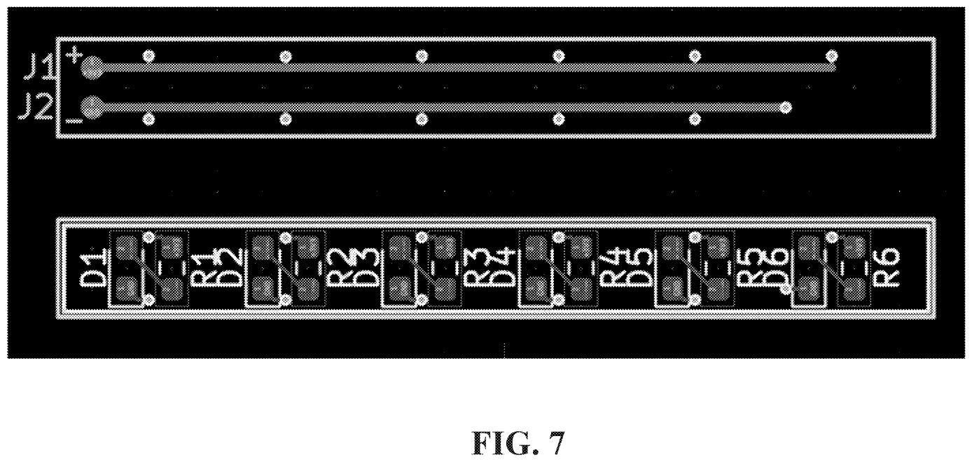

With reference to FIGS. 1-14C, a medical and dental camera, mobile application and monitoring system 100 is described herein. Because the camera, mobile application, and monitoring system are believed to have near-term use in connection within the dental field, the description that follows primarily focuses on use of the system for dental applications. However, the present invention is not intended to be limited thereto, and the present system is believed to have broad application throughout the medical field and even non-medical fields such as for in-home inspection and monitoring. Accordingly, any reference to use of the camera for dental purposes is intended for explanation purposes and the camera, mobile app, and overall system are not intended to be limited thereto.

### Camera

With reference to FIGS. 1-10, the intraoral camera may be intended for use with a mobile application designed to provide users with an efficient and reliable mechanism for monitoring oral health conditions. The mobile application may enable users to compare historical and current scans to observe longitudinal changes in oral health indicators. The camera may be a self-contained unit having a wand-shaped form factor.

In some example embodiments, the camera includes a lens assembly 104, an image sensor circuit 102, memory, and a processor. The image sensor 102 circuit may comprise, for example, a charge-coupled device (CCD) image sensor or, in other embodiments, a complementary metal-oxide semiconductor (CMOS) image sensor. In some CCD-based embodiments, the image sensor circuit may generate approximately 300,000-pixel image data.

In some example embodiments, the image sensor may comprise a CMOS image sensor, such as, by way of non-limiting example, a commercially available sensor having specification number OV2640 or an equivalent imaging sensor. Other embodiments may employ different CMOS or CCD image sensors having different resolutions, architectures, or interfaces.

In addition, the camera may include a rechargeable battery, wireless communication circuitry configured for Wi-Fi data transmission with the mobile application, and Bluetooth circuitry configured to support initial device pairing and Wi-Fi credential configuration.

In some embodiments, the camera may be powered by a high-capacity lithium-ion battery capable of providing up to four hours of continuous scanning on a single charge. In some embodiments, an example device supports fast charging via USB-C, allowing a full recharge in approximately 60 minutes. Additionally, in some embodiments, the system includes an intelligent power management feature that automatically enters sleep mode when idle, extending battery lifespan. Overheat protection mechanisms ensure safe operation, preventing excessive temperature buildup during prolonged use. Some embodiments may also support wireless charging, providing added convenience for users who prefer a cable-free experience. Battery capacity, charging duration, and operational time are illustrative and may vary based on implementation, usage patterns, and hardware configuration.

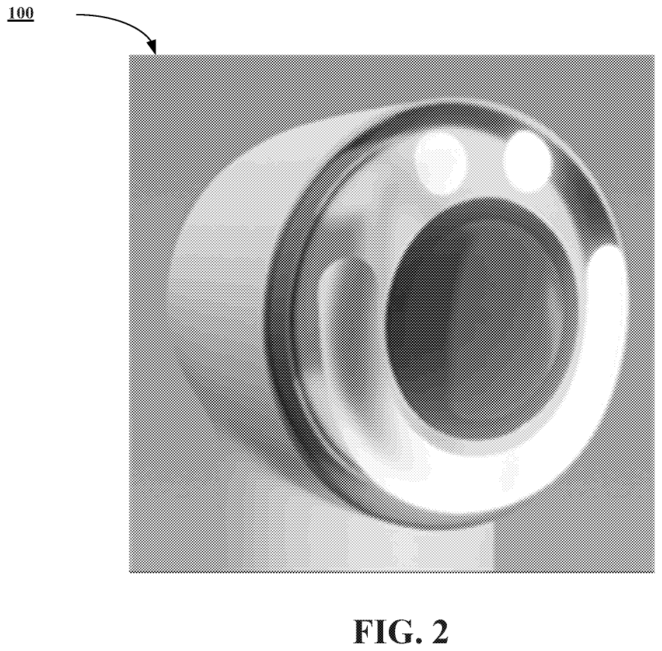

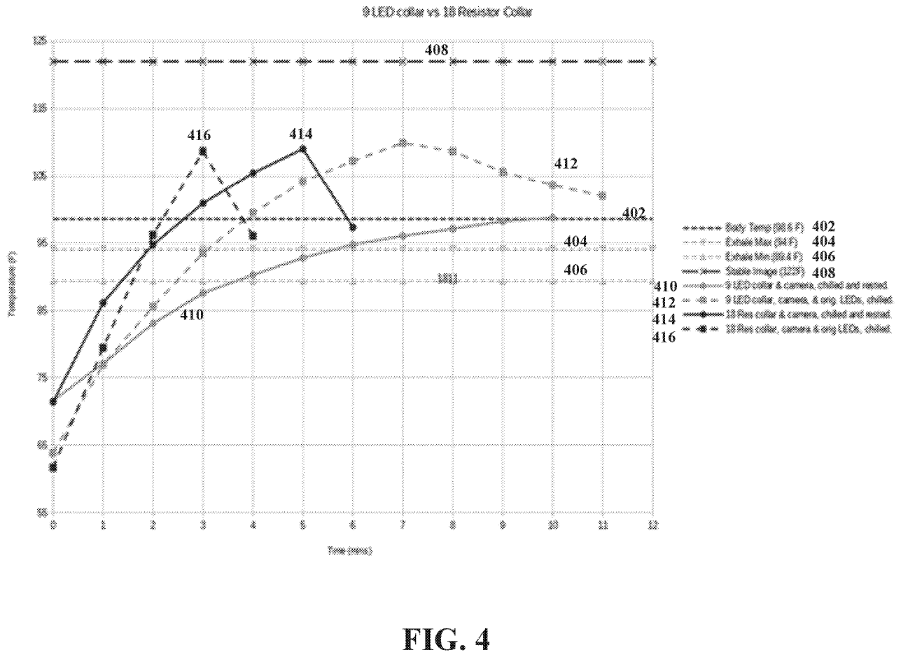

With reference to FIGS. 2-4, an improvement compared to prior intraoral cameras includes a pre-heating system 200 which reduces condensation (fogging) on the camera lens when the user places the camera in his/her mouth and breathes. The preheating system includes resistors which produce heat to bring the temperature of the lens close to the temperature of the user's breath to prevent condensation. When plugged in, the camera lens may be heated with a series of resistors until the camera lens reaches a temperature of 90° F.-100° F., with a preferred temperature of 94° F., after which the heating system turns off. A temperature sensor monitors the temperature of the lens and controls when to turn the heating system on and off. The camera may include any number of resistors to produce heat to minimize condensation. However, a preferred camera includes 10-25 resistors, a more preferred camera includes 15-20 resistors, and the preferred embodiment includes 18 resistors to uniformly heat the camera lens. Heating of the camera lens including 18 resistors and 9 light emitting diodes (“LEDs”), e.g., on LED flex PCB 106, the LEDs may be include 9 LEDs without resistors in FIG. 4. This diagram shows how the camera's heating system rapidly brings the temperature of the lens close to the exhale temperature of 89.4° F.-94° F. More specifically, the diagram illustrates how fast 18 resistors raise the temperature of the lens, versus how fast 9 standard LEDs would. Existing intraoral devices that incorporate anti-fogging features typically rely on lens geometry or surface characteristics, such as non-spherical lens profiles, rather than utilizing an active, temperature-controlled heating mechanism as implemented in the present system. The graph of FIG. 4 illustrates body temperature 402, exhake maximum temperature 404, exhale minimum temperature 406, stable image 408, 9 LED collar and camera chilled and rested 410, 9 LED collar and camera, and original LEDs chilled 412, 18 resistor collar and camera chilled and rested 414, and 18 resistors collar, camera, and original LEDs chilled 416. Although an example embodiment includes approximately 18 resistors, other embodiments may include fewer or greater numbers of heating elements, different heating element types, or alternative heating arrangements capable of reducing condensation. In some embodiments, the lens may be heated to a temperature approximately matching exhaled breath temperature, such as within a range of about 90° F. to about 100° F. Other embodiments may operate at different temperature ranges sufficient to reduce condensation.

With reference to FIGS. 5-7, the camera includes at least one, and more preferably between three and ten LEDs. In the preferred construction, the camera includes six LEDs to provide consistent light for imaging. The LEDs are positioned around the lens and facing at a 90-degree angle relative to the camera lens. The position and angle relative to the lens reduces glare. The camera's processor includes an onboard algorithm that adjusts the LED brightness in real-time based on the RGB and intensity histograms of the images captured, improving uniformity of image quality in all images produced.

### Lens Assembly

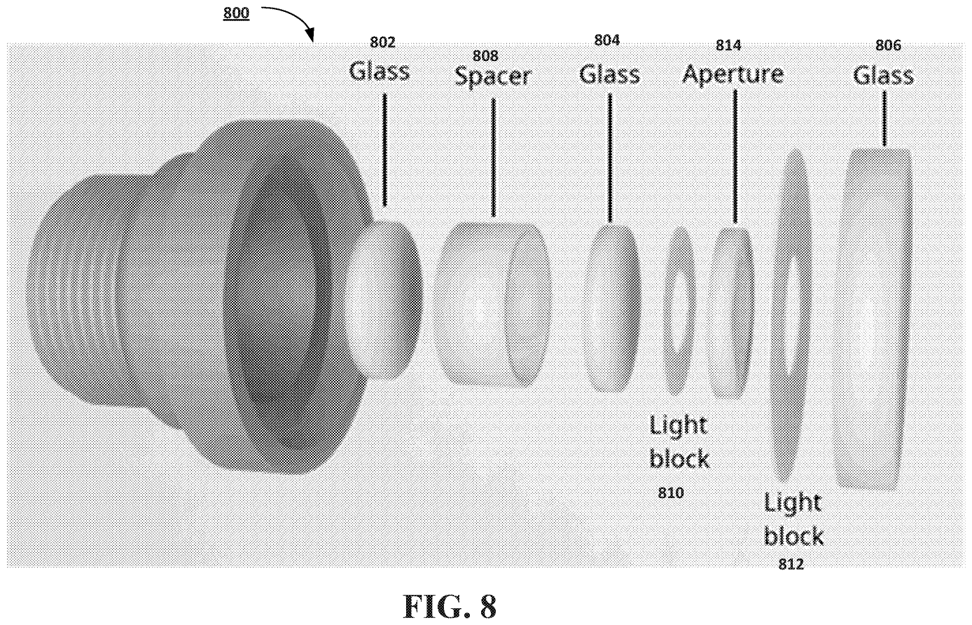

With reference to FIGS. 1 and 8, the lens assembly 800 includes several features that improve the images captured by the camera. The lens assembly includes three glass lenses 802, 804, 806, a spacer 808, an annular light block 810, 812, and an aperture 814. In addition, the lens assembly includes a diffuser 108 made of white vinyl which may be positioned in front of the LEDs to limit the glare while still providing adequate luminosity and may be protected by a clear plastic diffuser cover 110. Other devices that market anti-glare feature let the user adjust the brightness of the LED to reduce the glare. Conversely, the camera may automatically adjust the brightness of the LEDs, and an anti-glare filter may be placed in front of the LED to reduce any artifacts.

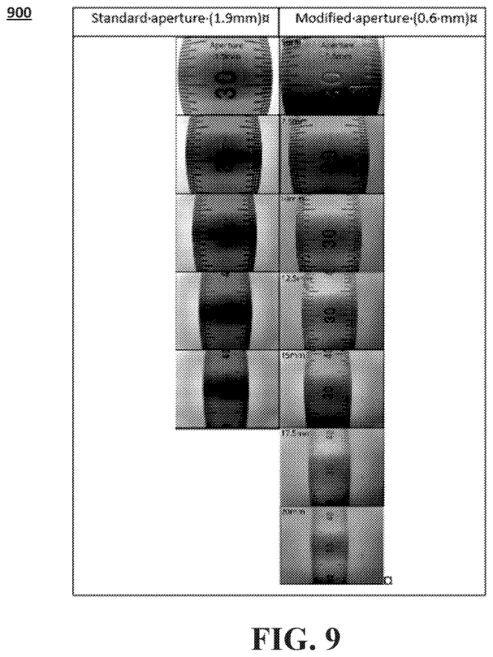

In addition, contrary to conventional dental cameras, in one example embodiment, the aperture ring of the lens has been reduced from 1.9 mm to 0.6 mm in order to increase the depth of field to 15 mm, which thereby extends the focus point from 5 to 20 mm. With reference to FIG. 9, by making this adjustment, users can easily take clear pictures 900 as the camera will produce sharp images when the lens may be positioned anywhere between 5 and 20 mm away from the object they are capturing. The illustrated aperture dimensions are provided by way of example only, and other aperture sizes or optical configurations may be employed to achieve comparable imaging performance.

In some example embodiments, the camera system may feature a modular design, allowing for the attachment of replaceable camera heads that extend the functionality of the device. Unlike fixed-lens intraoral cameras, some embodiments may enable users to swap out the camera head for different imaging modes.

In addition to interchangeable imaging heads, the camera system supports a replaceable toothbrush head attachment, allowing users to integrate oral hygiene functions directly into the scanning workflow. This modular design enables the camera to function as both an oral imaging device and a guided hygiene support device. In some embodiments, the system provides AI-based brushing guidance based on detected oral health condition indicators and usage patterns. The replaceable attachments enhance hygiene and convenience by permitting component replacement for extended device longevity and improved cross-user sanitation.

In some embodiments, the system includes a plurality of multi-use, replaceable imaging heads configured for selective attachment to a common handheld imaging device. The replaceable heads may include, by way of example and without limitation, intraoral camera heads for dental imaging, periodontal imaging attachments for gum health assessment, high-magnification lens assemblies for early detection of cavities and lesions, and fluorescence-based imaging attachments for visualization of plaque, demineralization, and tooth decay. Each replaceable head may be mechanically and electrically coupled to the handheld device through a standardized interface, thereby enabling rapid interchangeability while maintaining consistent image acquisition, illumination control, and data transmission performance. The listed imaging heads are exemplary, and additional or alternative imaging attachments may be provided without departing from the scope of the disclosure.

The modular camera head system allows users to interchange imaging modules based on selected imaging objectives. Each attachment employs a precision locking mechanism that provides secure mechanical coupling while maintaining optical alignment. When a camera head is replaced, the system may automatically recalibrate focus, illumination, and exposure parameters to maintain imaging consistency.

The modular heads may further incorporate an auto-recognition feature that enables the mobile application to adjust imaging settings based on the identified head type. For example, high-magnification attachments may trigger modified focal depth and exposure settings to improve visualization of fine surface features, while fluorescence-based heads may activate specific illumination wavelengths to enhance visualization of surface condition indicators such as plaque accumulation or enamel demineralization patterns.

In contrast to fixed-lens intraoral cameras, this modular architecture supports hardware extensibility by permitting future imaging capability upgrades without replacement of the entire device system.

### Electrical Circuitry Optimization

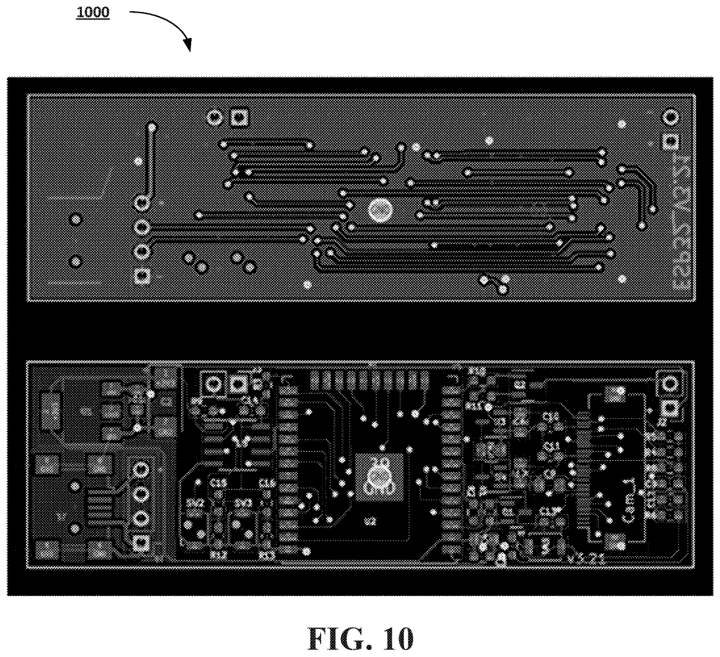

The camera's circuit design may be optimized to reduce interferences and provide uninterrupted Wi-Fi connectivity with the mobile application. The location of the components on the PCB may be tailored to minimize interferences. With reference to FIG. 10, in the illustrated example, the placement of the components on the PCB 1000 eliminates the need for wires, which are known to generate interference. In particular, the power button may be designed to facilitate direct contact with the electromechanical components on the Printed Circuit Board (PCB) without wires. In addition, the Printed Circuit Board may be made of a flexible material (Flex PCB) to fit within the neck of the intraoral camera to minimize its distance from the camera lens. The Wi-Fi antenna may be placed on the back of the camera, isolated from the rest of the component with Polyimide Film.

In some embodiments, the system implements a multi-layered artificial intelligence (AI)-based analysis framework configured to analyze, classify, and longitudinally track oral health condition indicators using captured intraoral image data. The AI framework may operate in a plurality of sequential processing layers, each layer refining the image data to ensure that only clinically relevant and diagnostically informative images are retained for downstream processing and assessment.

The described processing layers are illustrative. In other embodiments, the functions of the layers may be combined, reordered, implemented in parallel, or omitted, or may be implemented using different model architectures, while achieving similar filtering, identification, and longitudinal tracking functionality.

A first processing layer may include a classifier model configured to determine whether a captured image corresponds to a valid intraoral scan or an irrelevant capture, such as images of the tongue, lips, cheeks, or external facial regions. Images determined to be non-diagnostic may be automatically excluded from further analysis.

A second processing layer may include a blurriness detection model configured to evaluate image sharpness, focus quality, and motion artifacts. Images that fail to satisfy a predefined clarity threshold may be discarded or flagged for reacquisition, thereby ensuring that only images meeting minimum quality requirements are processed.

A third processing layer may include a tooth number identification model configured to identify, segment, and label individual teeth within each retained image. The labeled tooth data may be used to eliminate redundant image captures, associate images with specific tooth identifiers, and optimize longitudinal tracking of dental pathology, including cavities, lesions, plaque accumulation, and periodontal conditions.

In some embodiments, the outputs of the respective processing layers may be stored in association with corresponding patient records, enabling automated comparison across multiple imaging sessions to detect temporal changes in oral health status.

Once an image passes through these layers, a segmentation model highlights anatomical features such as teeth, gums, and potential pathological regions. Using deep learning techniques, the system precisely outlines regions corresponding to decay indicators, plaque accumulation patterns, gingival inflammation markers, and enamel erosion features for better visualization by users and dental professionals. In other embodiments, similar functionality may be implemented using fewer, additional, or alternative model types.

The final pathology tracking model enables long-term monitoring of dental conditions. Historical images are compared against new scans to assess changes in decay, gum recession, calculus buildup, and enamel wear. This AI-driven comparison alerts users to early warning signs, encouraging timely intervention before conditions worsen.

The Transmission Control Protocol/Internet Protocol (TCP/IP) connection settings are configured to augment the buffer size on the camera. This may be optimize the transmission of the video stream and enhance the communication with the mobile application. The Wi-Fi transceiver within the camera can connect to any Wi-Fi router to transmit the oral scans seamlessly to any smart devices. In the event that a Wi-Fi network is not available, the camera can also directly connect to the smart device to transmit the scanned data such as by using other wired or wireless connections, such as Bluetooth or a USB cable connection.

The described communication protocols are provided by way of example only. Other wired or wireless communication mechanisms, protocols, or transport layers may be used to transmit image data and control signals between system components.

The camera system may be designed for long-term durability, incorporating impact-resistant materials and a reinforced lens coating to prevent scratches. The housing may be constructed from medical-grade polymer with shock-absorbing properties, allowing the device to withstand accidental drops from typical usage heights. The lens features an anti-scratch coating that preserves optical clarity over time, ensuring consistent image quality. Additionally, in some embodiments, the camera may be certified IPX7 waterproof, enabling full immersion in disinfectant solutions for thorough sanitation. These durability enhancements make the system suitable for both home and clinical environments, ensuring reliability under repeated use.

The AI-based analysis framework may be trained using a combination of labeled reference datasets and real-world intraoral scan images. Model performance may be refined through periodic retraining using anonymized image data in accordance with applicable privacy controls. The AI classifier may be configured to filter out non-relevant or low-quality images by referencing a training set of labeled intraoral image examples, thereby ensuring that primarily clinically informative image data may be retained for processing. To reduce classification error, the system may employ an adaptive training loop in which incorrectly categorized images are flagged for additional review and incorporation into subsequent training iterations. In addition, AI-based image segmentation identifies anatomical landmarks, including individual teeth and gingival structures, to improve consistency and reliability of image analysis outputs.

In some embodiments, the camera system may be waterproof, achieving an IPX7 rating to enable safe disinfection and hygienic use in home and clinical settings. Unlike traditional intraoral cameras, which require protective covers or delicate cleaning procedures, some example embodiments may allow direct immersion in disinfectant solutions or running water cleaning.

The waterproofing technology may be achieved through a sealed housing with polymeric coatings and gasket-sealed electronic components that prevent moisture ingress while maintaining full image clarity and device durability.

### Mobile Application and Image Processing

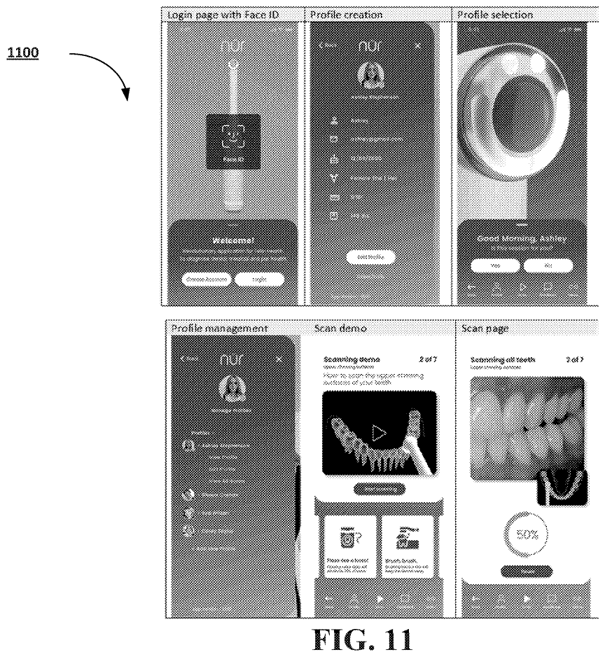

Contrary to other intraoral cameras, some example cameras described herein may capture the whole oral scan which may be transmitted in real-time to a smart device, such as a mobile phone or tablet. With reference to FIG. 11, as the user scans his/her mouth, the application displays the live video real-time on their application installed smart device which has been transmitted by Wi-Fi from the camera to the smart device, such as their mobile phone or tablet. Artificial Intelligence (“AI”) processing analyzes the full video instead of a couple of images, giving a holistic view of the user's oral health 1100. User interface flows and guidance sequences are illustrative and may be modified, reordered, or omitted in other embodiments.

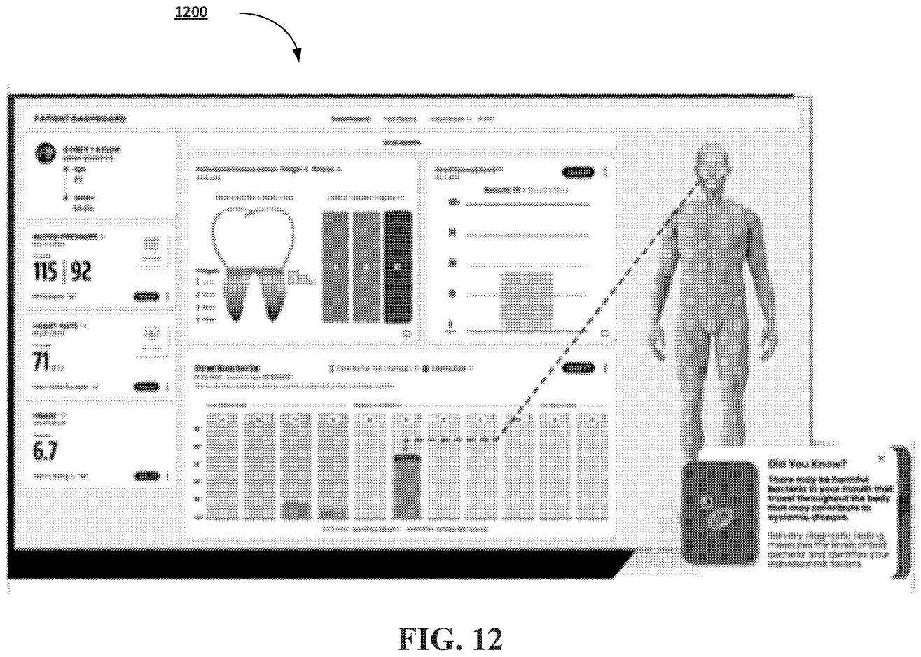

FIG. 12 illustrates an example patient dashboard 1200. In some embodiments, the system's Mouth-Body Connection® feature extends oral imaging analysis by correlating intraoral image data with systemic health-related indicators. Using AI-based pattern analysis, the platform evaluates salivary biomarker information, monitors longitudinal trends in gingival recession, and analyzes bacterial presence, and cross-references these data with curated medical research datasets to generate informational correlation outputs.

In some implementations, the system identifies potential associations between oral condition indicators and systemic health risk factors. For example, chronic gingival inflammation and periodontal condition indicators may be associated with elevated cardiovascular risk factors; variations in gingival bleeding and healing response may be associated with glycemic control indicators; and detection of certain oral bacterial patterns may be associated with neuroinflammatory risk correlations. These correlations are provided for informational and risk-awareness purposes and are not intended to replace professional medical diagnosis or treatment.

Based on these analyses, the system generates an AI-driven systemic risk score that may be displayed on the user dashboard. This real-time score alerts users to potential medical concerns, prompting them to seek further professional consultation as needed.

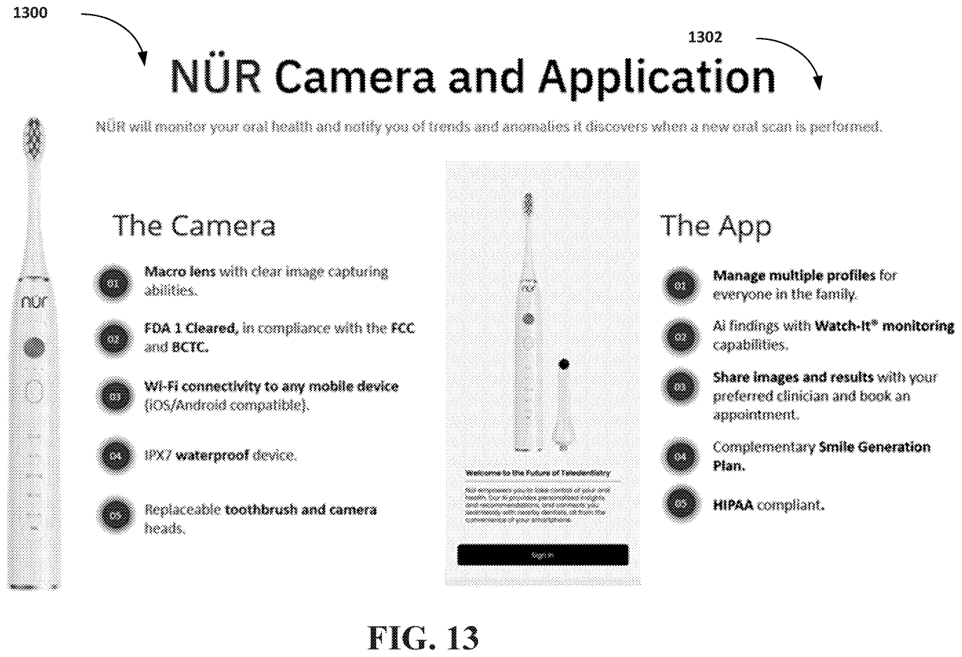

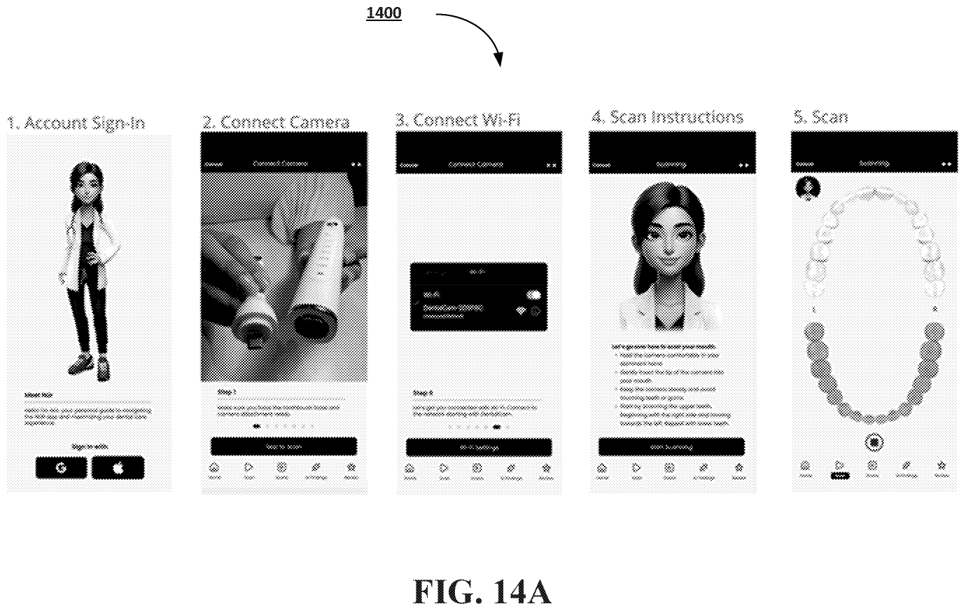

FIG. 13 illustrates the camera 1300 and applications 1302. In select embodiments, the system can synchronize with wearable health devices and electronic health record (EHR) systems, enabling seamless collaboration between dental and medical professionals. By integrating intraoral scan data with broader health records, the AI-generated risk assessment provides predictive insights that empower users to take preventative action before oral conditions impact overall health.

### Multi-Layered AI Analysis Framework

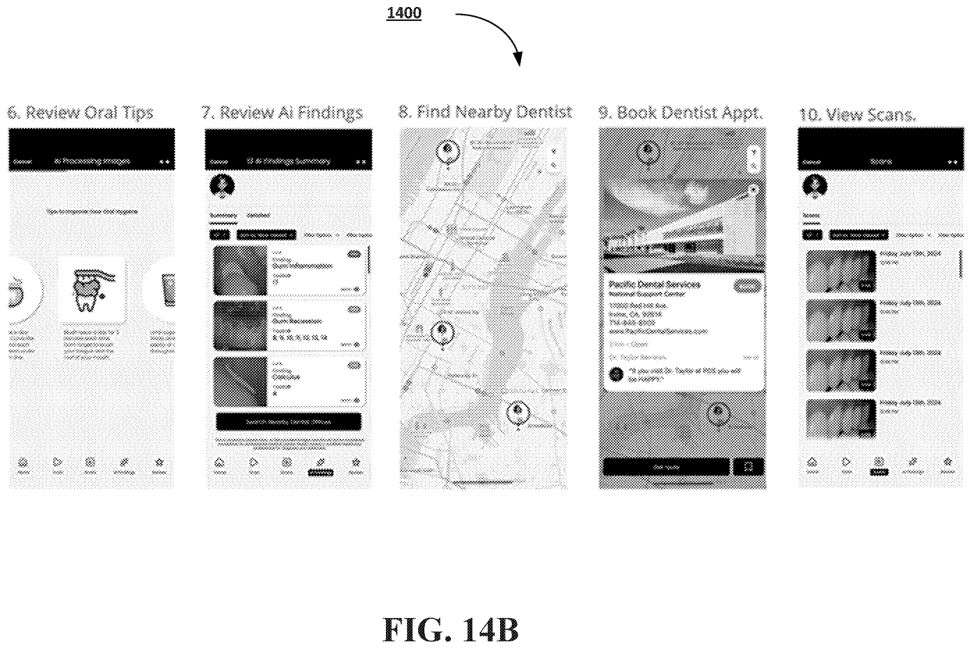

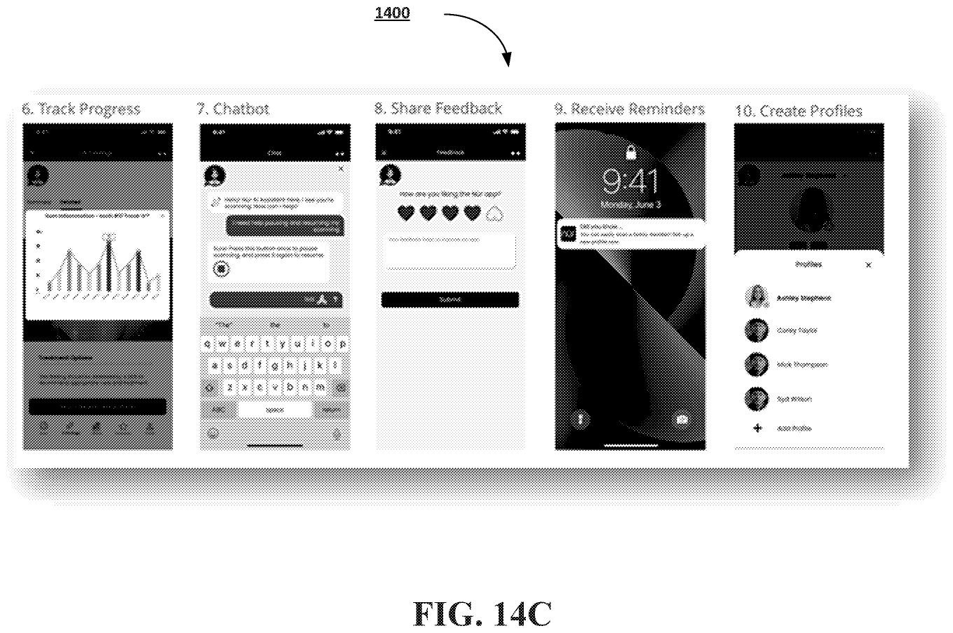

FIGS. 14A-14C illustrate an example user interaction and processing flow implemented 1400 by the camera system. In this embodiment, the camera system employs a multi-layered artificial intelligence (AI) analysis framework configured to efficiently analyze and monitor oral health conditions. The AI framework operates sequentially in three processing layers to ensure that only clinically relevant images are retained for further image analysis.

A first processing layer includes a classifier model configured to determine whether a captured image qualifies as a valid intraoral scan by filtering out non-clinical content, including images of soft tissue, facial regions, or external environments.

A second processing layer includes a blurriness detection model configured to evaluate image sharpness, focus accuracy, and motion distortion. Images that do not meet a minimum clarity threshold may be automatically discarded or flagged for reacquisition, thereby reducing the likelihood of distorted or unreliable image analysis results.

A third processing layer includes a tooth number identification model configured to assign identifiers to individual teeth within each retained image. The assigned identifiers enable subsequent analyses to target correct anatomical regions while eliminating redundant imaging of the same tooth structures.

In some embodiments, the outputs of the three processing layers are combined to generate a refined image dataset that may be stored in association with user or patient records and used for longitudinal monitoring, pathology indicator identification, and clinical reporting support.

### Real-Time Augmented Reality Guidance

To enhance usability, the mobile application integrates real-time augmented reality (AR) overlays. These overlays provide visual cues—such as optimal positioning, angle adjustments, and haptic feedback (with optional voice-guided instructions)—to assist users in capturing high-quality scans. This guided scanning process minimizes user error and ensures consistent imaging across sessions.

AI-Driven Pathology Detection and Risk Assessment

Following initial image filtering and quality validation, the system applies a deep learning-based segmentation model configured to precisely delineate anatomical features and potential pathological regions within the intraoral images. The segmented regions may include, by way of example and without limitation, areas of tooth decay, plaque accumulation, gingival inflammation, and enamel erosion. The segmentation outputs may be stored as region-specific masks, contours, or coordinate sets associated with each image.

In some embodiments, the framework further incorporates a dedicated pathology tracking layer configured to compare historical intraoral scans with newly acquired images for a same patient. The pathology tracking layer may detect subtle temporal changes, including increases in lesion size, progression of decay, changes in gingival margins, or variations in plaque density, and may generate proactive alerts recommending early clinical intervention.

In further embodiments, the system cross-references the intraoral scan data and pathology tracking results with established systemic health correlations stored in a medical knowledge database. Based on the cross-referencing, the system may identify early potential indicators associated with systemic conditions, including, without limitation: cardiovascular disease, in which chronic periodontal inflammation may be correlated with increased risk of atherosclerosis and stroke; diabetes, in which variations in gingival bleeding, tissue healing rates, or inflammatory markers may indicate suboptimal glycemic control; neurodegenerative diseases, in which detection of specific oral bacterial strains may be associated with neuroinflammation and cognitive decline; and pneumonia risk, in which elevated bacterial accumulation within the oral cavity may increase the likelihood of aspiration pneumonia, particularly in elderly or immunocompromised patients.

In some embodiments, the identified systemic risk indicators are presented to a clinician or patient through a user interface together with confidence scores, explanatory correlations, and recommended follow-up actions.

### System Integration and Predictive Analytics

Based on these analyses, the system generates an AI-driven systemic risk score displayed on the user dashboard, alerting users to potential medical concerns and prompting timely professional consultation. In select embodiments, the system can also synchronize with wearable health devices and electronic health record (EHR) platforms to enable comprehensive, collaborative care between dental and medical professionals.

In an example embodiment, the AI processing may utilize one or more machine-learning frameworks, such as TensorFlow converted to quantized CoreML to allow the model to run efficiently on a mobile phone. Tensor flow is a framework of machine learning and deep learning developed by the Google Brain Team. It may be based on Python programming language and use for numerical computation and data flow, which makes machine learning faster and easier. Advantageously, TensorFlow may be capable of running in Android, IOS and Web based applications. Meanwhile, Core ML is an Apple developed system that uses a machine learning algorithm to a set of training data to create a model. The model then makes predictions based on new input data. Advantageously, the Core ML models can accomplish a wide variety of tasks that would be difficult or impractical to write in code. For example, you can train a model to categorize photos, or detect specific objects within a photo directly from its pixels. This TensorFlow and Core ML combination of AI and machine learning has been found to be useful in some embodiments for processing oral images. The specific machine-learning framework used to implement the AI models is not critical, provided that the framework supports execution of trained models for image classification, segmentation, or analysis.

The mobile application supports remote consultations with dental professionals by enabling secure image transmission via an encrypted cloud-based platform. Users may transmit intraoral scans to dentists or AI-assisted telehealth platforms to receive real-time analytical feedback and professional evaluation support without requiring an in-office visit.

In some implementations, the system generates AI-based analysis reports that provide risk assessments and follow-up guidance based on analyzed intraoral scan data. The reports may include quantitative severity scores, progression indicators, and prioritized condition findings. In some embodiments, dentists or other authorized clinicians may review the AI-analyzed findings and provide virtual consultations to patients through an integrated tele-dentistry portal, thereby supporting remote clinical evaluation, care planning, and follow-up monitoring.

In some embodiments, the tele-dentistry and analysis platform further supports secure data management and longitudinal monitoring. Features may include HIPAA-compliant encrypted data transmission, cloud-based storage for historical scan tracking and progressive condition indicator analysis, and automated risk scoring with corresponding follow-up guidance generated by the AI analysis framework. Historical datasets may be used to identify condition progression patterns, care response trends, and user-specific risk indicators over time.

To ensure privacy, regulatory compliance, and data security, the system implements multiple security layers. In some embodiments, all stored images and associated patient data are protected using Advanced Encryption Standard 256-bit (AES-256) end-to-end encryption. Data transmitted between the camera device, mobile application, and cloud servers may be secured using Transport Layer Security (TLS) version 1.3 encryption. Secure user access may be enforced through multi-factor authentication (MFA). Additionally, in some implementations, local AI processing may be performed on the camera device or mobile device to reduce reliance on cloud servers, thereby enhancing data privacy, reducing transmission latency, and minimizing exposure of sensitive patient information.

Users have the option to store scans locally on their device or opt for HIPAA-compliant cloud storage for long-term tracking and tele-dental consultations. In offline scenarios, the camera enables direct peer-to-peer Wi-Fi transmission, ensuring full functionality even in areas with limited internet access.

This telehealth functionality enhances accessibility, making professional dental monitoring available to underserved populations, remote users, and those seeking preventative care.

Thus, the camera, smart device and App combination provides an example of AI capabilities that may be run on any smart device. Real-time AI processing takes place on the user's smart device (mobile phone, tablet, etc.) as the scan may be happening. Advantageously, software in the camera and the application software in the smart device provide parallel processing to achieve two or more tasks simultaneously. Specifically, the camera processes and live-stream the images of the oral scan to the smart device. The mobile device then conducts AI processing of each image as they are being received. Multiple layers of Artificial Intelligence (AI) maximize the computation resources available on the user's phone by processing only the most relevant information. These steps may include removing the non-clinical or out of focus images. The processor then automatically conducts tooth numbering within each image, and an image comparison model eliminates redundant information. From this information, the mobile device identifies and tracks pathologies and aesthetic concerns in the user's mouth. These include: decay, calculus, gum recession, gum inflammation, and stain and yellowing.

The system may integrate with HbA1c monitoring, Oral FitnessCheck®, and Oral DNA® testing to analyze salivary biomarkers and provide a more comprehensive assessment of patient health. The AI processes oral imaging results and cross-references them with known systemic health correlations to detect early warning signs of disease.

The system may be designed to integrate seamlessly with electronic health record (EHR) platforms, including Epic Cosmos and other healthcare data management systems. Through this integration, AI-generated oral health insights can be linked to a patient's broader medical history, enabling physicians and dental professionals to collaborate on preventative and interventional care strategies. By cross-referencing intraoral scans with systemic health data, the reliability of AI-assisted image assessment, supports early disease detection, and provides a comprehensive overview of patient health. Additionally, users may authorize secure data sharing with their healthcare providers, ensuring that both dental and medical professionals have access to AI-driven insights that support personalized treatment plans.

Some implementations provide a patient dashboard that visually maps oral health changes over time and their potential systemic impact. The dashboard may integrate AI-generated risk scores, suggest preventative actions, and provide personalized health recommendations based on detected trends.

By integrating systemic health monitoring, the application can identify oral indicators that may correlate with broader medical conditions. For example, chronic gum inflammation (periodontitis) may be linked to an increased risk of heart disease. The AI-driven analysis can provide risk assessments based on oral health conditions and suggest appropriate follow-ups with medical professionals.

Some embodiments may incorporate a conversational chatbot designed to assist users with common dental inquiries and provide real-time feedback on scan results. The chatbot may leverage AI-based natural language processing to answer questions related to oral hygiene, interpret scan data, and offer personalized recommendations. Additionally, it may provide reminders for brushing, flossing, and dental appointments, to help ensure users maintain consistent oral care habits. In an example embodiment the chat bot may answer questions on the application (app) such as how to use app, the purpose of the app, troubleshoot the app, or other questions about the app.

In some implementations, the system may analyze flossing habits by evaluating how effectively users clean interdental spaces. The AI-based model may assess the presence of plaque buildup or gum irritation in areas that indicate improper flossing technique. By tracking flossing-related health indicators over time, the system may provide insights into whether a user's flossing habits are improving and offer recommendations for more effective flossing.

The AI-powered system tracks brushing and flossing habits over time, analyzing motion patterns, pressure, and frequency to provide real-time feedback on technique effectiveness. Unlike standard tracking methods, which rely solely on manual input, the AI continuously evaluates the consistency and completeness of oral hygiene routines.

In some embodiments, the system further includes habit-tracking and longitudinal monitoring features configured to evaluate user behavior and oral hygiene trends based on repeated intraoral scans. For example, a tooth staining analysis function may compare image coloration values over time to detect shifts in tooth shade, thereby enabling users and clinicians to monitor whitening treatment effectiveness, dietary staining impact, or enamel health changes.

In some embodiments, a bruxism detection function analyzes enamel wear patterns, surface flattening, edge chipping, and microfractures to identify indicators associated with nighttime or stress-related teeth grinding. When such patterns exceed predefined thresholds, the system may generate alerts recommending clinical evaluation, night-guard usage, or behavioral interventions.

In further embodiments, a plaque accumulation monitoring function applies AI-driven pattern recognition to identify plaque distribution, density, and progression trends across successive scans. Based on the detected trends, the system may generate targeted hygiene recommendations, including region-specific brushing guidance, flossing emphasis, or professional cleaning reminders.

In some implementations, the habit-tracking outputs are integrated with the pathology tracking and risk assessment modules to provide a comprehensive, behavior-aware oral health profile for each user.

These behavioral insights enable users to proactively adjust their hygiene routines and prevent the progression of oral health conditions before they require professional intervention.

In certain embodiments, the system may analyze dental pathologies, such as cavities, gum disease, enamel erosion, and other abnormalities. The AI-driven detection system may compare images from previous scans to track the progression of dental conditions over time. Users may receive visual representations of how conditions are changing, enabling proactive intervention and facilitating remote monitoring by dental professionals.

Some embodiments may integrate a location-based service that allows users to find nearby dentists directly within the mobile application. The system may use GPS and local directories to provide a list of dental professionals within a predefined radius, along with their contact information, ratings, and availability. This feature may streamline the process of scheduling in-person consultations when necessary.

In some implementations, the mobile application may integrate with electronic health record (EHR) systems such as Epic to facilitate seamless scheduling of dental appointments. Users may be able to book appointments directly from the app, reducing the need for manual coordination. Additionally, appointment reminders may be sent via notifications to ensure users stay on track with their dental visits.

Some embodiments may allow users to securely transmit captured images to their dentist for remote evaluation. The system may support encryption protocols to ensure HIPAA-compliant data transmission, allowing dental professionals to review scans and provide feedback without requiring an in-office visit. This feature may enhance telehealth capabilities and improve access to professional dental guidance.

In some embodiments, the system provides functional and usability improvements relative to conventional intraoral cameras by enhancing image analysis reliability, workflow efficiency, and user engagement. Unlike traditional intraoral cameras that primarily capture and store images, the disclosed system integrates real-time AI-based analysis to generate immediate, user-accessible insights regarding oral health condition indicators.

Some examples may include a Watch-It monitoring feature, which enables users to track specific oral health concerns over time. Unlike prior systems that require manual image comparison, Watch-It automates the tracking of dental pathologies, such as cavity expansion, gum recession, and enamel degradation. The system provides proactive alerts if a monitored condition worsens, prompting users to seek professional intervention.

In some embodiments, the system may be configured with modular adaptability to support future hardware and software upgrades without requiring replacement of a core imaging device. By way of example and without limitation, the modular architecture may accommodate specialized fluorescence-based attachments for enhanced decay detection, hyperspectral imaging modules for early-stage cavity identification based on wavelength-specific reflectance characteristics, and periodontal imaging adapters configured for deep tissue visualization and subgingival analysis.

In some implementations, each modular upgrade component may be mechanically, optically, and electrically interfaced with the core device through a standardized coupling interface, thereby enabling backward compatibility, simplified integration, and continued expansion of imaging and analysis capabilities as imaging technologies evolve.

This modular approach may ensure the camera system remains adaptable to emerging dental technologies, differentiating it from fixed-function competitors.

In some implementations, the mobile application may provide a library of educational videos or frequently asked questions, covering oral hygiene best practices, proper brushing and flossing techniques, and information on common dental conditions. These videos may be tailored to individual users based on their scan results, ensuring they receive relevant and practical guidance for maintaining optimal oral health.

In some embodiments, the systems and methods described herein are configured to integrate with value-based dental insurance programs and preventative care incentive platforms. The integration enables users to earn incentives based on consistent oral health monitoring and participation in AI-driven preventative care tracking. For example, users may qualify for reduced insurance premiums based on maintaining a predefined scan frequency, expanded preventative care coverage determined from AI-detected oral health trends, and eligibility for rewards-based oral health programs in which demonstrated dental hygiene compliance results in financial, service-based, or coverage-related benefits.

In some implementations, the system transmits compliance metrics, anonymized health indicators, or summarized risk scores to participating insurance or benefits platforms in accordance with applicable privacy regulations. The incentive determinations may be dynamically updated based on longitudinal scan data, pathology risk trajectories, and behavioral compliance metrics, thereby encouraging proactive oral health management while supporting preventative, outcome-based insurance models.

The system may be designed to support value-based healthcare reimbursement models by integrating preventative oral health tracking with dental and medical insurance programs. Through secure AI-driven analysis, the system generates compliance reports that document a user's oral health trends, preventative care adherence, and early pathology detection rates. These reports can be shared with insurers and healthcare providers to qualify users for preventative care benefits, reducing overall healthcare costs by identifying and mitigating conditions before they require invasive treatment.

In some embodiments, the system may be configured to interface with insurance providers that implement value-based reimbursement models. Under such models, users who maintain consistent oral health monitoring using the disclosed system may qualify for reduced out-of-pocket costs for dental procedures, expanded preventative care coverage including subsidized professional cleanings and clinical evaluation visits, and insurance premium adjustments based on demonstrated improvements in oral health indicators derived from AI-tracked scan data.

In some implementations, eligibility determinations are generated automatically using compliance metrics, pathology risk trajectories, and longitudinal oral health indicators produced by the AI analysis framework. The system may securely transmit eligibility status, anonymized performance indicators, or incentive qualification confirmations to participating insurance platforms in accordance with regulatory and privacy requirements.

The system automatically logs scan frequency and pathology progression, generating insurance compliance reports that dental providers can use to customize patient coverage. This incentivized approach improves patient engagement and encourages long-term preventative care compliance.

Certain embodiments may include automated notifications to remind users to perform regular scans and track oral health progress. Additionally, the system may prompt users to create profiles for family members, ensuring that all household members benefit from routine monitoring and preventative care insights.

In some embodiments, the system incorporates gamification features configured to encourage consistent oral health monitoring and sustained user engagement. The gamification features may include daily and weekly personalized challenges generated based on AI-detected oral health trends, such as targeted brushing improvement, plaque reduction, or scan frequency goals.

In some implementations, the system maintains progress-tracking streaks that record consecutive days or weeks of completed scans, hygiene compliance, or challenge participation, thereby incentivizing long-term adherence to recommended oral care routines.

In further embodiments, the system generates AI-based achievement indicators, including digital badges, milestone awards, and streak counters, which are issued when users demonstrate measurable improvements in brushing technique, reductions in plaque levels, or consistent scan frequency over predefined time intervals.

In some implementations, the gamification outputs are integrated with the analysis, habit-tracking, and incentive platforms described herein, thereby providing a behavior-reinforcement mechanism that supports preventative care engagement, compliance motivation, and longitudinal oral health monitoring.

By incorporating these elements, the system fosters habitual engagement and ensures users remain proactive in their dental care routines.

Some embodiments may incorporate AI-powered smile simulation filters that allow users to preview potential dental treatments in real-time. These filters may include braces simulation, veneers simulation, teeth whitening previews, and crooked teeth correction visualizations. By using augmented reality (AR) technology, the system may help users make informed decisions regarding cosmetic and orthodontic procedures.

### Advanced Mobile Application Features

In some embodiments, the system includes a mobile application configured to enhance user experience, safeguard data security, and provide seamless integration with healthcare information systems. The mobile application may implement HIPAA-compliant data handling using end-to-end encryption and secure cloud storage to protect all captured intraoral scans and associated patient information in accordance with applicable regulatory requirements.

In some implementations, the mobile application interfaces with one or more electronic health record (EHR) systems, including, by way of example and without limitation, Epic Cosmos or equivalent platforms. The EHR integration enables collaborative review of oral health data by dental and medical professionals, thereby supporting coordinated patient care, interdisciplinary clinical evaluation, and streamlined clinical workflows.

In further embodiments, the mobile application provides automated reminders and engagement features. The application may monitor scan frequency and generate notifications prompting routine oral health checks. Additional reminders may be configured for hygiene activities, including brushing or flossing at daily, twice-daily, or other user-defined intervals. The engagement features may further incorporate gamification elements such as progress tracking, achievement badges, streak indicators, and AI-powered feedback insights to promote long-term adherence.

In some embodiments, the mobile application generates personalized health reports based on AI analysis of longitudinal scan data. The reports may include trend visualizations, risk indicators, behavioral correlations, and explanatory insights, thereby enabling users to monitor oral health progression and make informed preventative care decisions over time.

### Augmented Reality (AR) Guidance

Real-time AR overlays provide dynamic, on-screen guidance during scanning by highlighting areas needing attention. These smart visualization techniques ensure optimal image capture and assist users in maintaining proper technique.

### AR-Driven Functionalities

In some embodiments, the system further includes augmented reality (AR) visualization features configured to enhance user understanding, engagement, and treatment planning. For example, a whitening simulation filter may apply an AR-based enhancement overlay to captured intraoral images to provide a simulated preview of whiter teeth. This allows users to visualize potential outcomes of whitening treatments prior to undergoing a clinical procedure, thereby improving motivation and informed decision-making.

In some implementations, the system provides an AI-powered brushing simulation in which live AR overlays are rendered on scanned teeth to identify regions requiring additional brushing or improved technique. The overlays may dynamically update based on detected plaque distribution, enamel condition, or brushing history.

In further embodiments, smile simulation filters enable users to preview potential orthodontic or cosmetic treatments, including, by way of example and without limitation, teeth whitening, braces, aligners, veneers, or other restorative modifications. The simulated renderings may be generated using AI-based facial and dental alignment models to maintain anatomical accuracy.

In some embodiments, decay and plaque visualization features display real-time AR overlays highlighting detected plaque accumulation, demineralization regions, and decay spots, thereby enabling immediate identification and targeted hygiene or treatment actions.

In some implementations, the AR visualization outputs are integrated with the analysis, habit-tracking, and reporting modules described herein to provide an interactive, educational, and clinically supportive user experience.

### Data Security and Privacy

To comply with HIPAA and international data security standards, all image and scan data are protected by end-to-end encryption. Data are stored in an encrypted cloud environment with robust access control, and local AI processing is supported so that sensitive information need not be transmitted externally unless explicitly permitted. A zero-knowledge encryption framework further ensures that users retain full control over their data.

### AI-Powered Compliance Tracking

The system integrates with dental insurance providers by generating automated, HIPAA-compliant compliance reports that reflect scan frequency and pathology tracking. These reports help insurers assess adherence to preventative care protocols.

### Insurance-Linked Benefits and Value-Based Care

In some embodiments, the system supports a value-based care model using AI-driven oral health tracking and compliance analytics. Under this model, participating users may receive a complimentary imaging device in connection with enrollment in a participating dental or healthcare insurance plan. Users who maintain routine scan compliance may qualify for reduced insurance premiums, while those demonstrating measurable improvements in oral health through longitudinal AI analysis may receive expanded preventative care coverage.

In further implementations, the system enables performance-based reward programs in which financial, service-based, or coverage-related incentives are issued based on AI-verified compliance with recommended hygiene practices, scan frequency targets, and pathology risk reduction trends. The compliance verification may be derived from scan consistency metrics, plaque reduction indicators, enamel condition stabilization, and periodontal improvement trajectories.

In some embodiments, the value-based care determinations are dynamically updated using continuously accumulated AI tracking data, thereby promoting preventative care engagement, improving long-term outcomes, and aligning patient incentives with clinically meaningful oral health improvements.

In some embodiments, the system may be configured to integrate with insurance provider networks to support incentive-based preventative care programs. By consistently using the intraoral camera and associated application to perform regular oral scans, users may qualify for reduced insurance premiums, expanded preventative dental care coverage, and access to AI-based oral health risk scoring programs.

In some implementations, qualification status may be determined using longitudinal compliance metrics, pathology trend analysis, and behavioral indicators generated by the AI analysis framework. The system may securely transmit eligibility confirmations, anonymized risk indicators, or compliance scores to participating insurance platforms in accordance with applicable privacy and regulatory requirements.

In further embodiments, the incentive programs dynamically adjust benefits based on continued scan adherence and oral health improvement trajectories, thereby reinforcing preventative care participation and long-term outcome optimization.

Some embodiments track user compliance with recommended oral health habits, rewarding users who maintain regular scan frequency and good oral hygiene with financial benefits from their insurance providers.

The insurance compliance tracking feature can generate reports for value-based insurance models, allowing dental plans to customize coverage based on real-time patient data.

By incorporating secure cloud backup, real-time AI-based analysis, and interoperability with healthcare information systems, the systems and methods described herein provide an integrated intraoral imaging and monitoring platform that supports clinical review, longitudinal tracking, and patient engagement.

Some embodiments may allow users to create multiple profiles within the mobile application, enabling families to track the oral health of each member separately. Each profile may store scan history, AI analysis results, and progress reports, ensuring personalized recommendations and monitoring for every individual in the household.

In some embodiments, the systems and methods described herein are configured to support compliance with applicable regulatory requirements for medical and dental imaging devices. The system may be classified under medical device regulatory frameworks and may be designed to undergo appropriate certification, validation, and quality control processes to support safe and reliable operation in clinical and home environments.

In some implementations, the system may support periodontal fitness tracking and oral fitness assessment features that enable users to evaluate gum-related condition indicators using compatible external measurement tools. The application may further support integration with third-party periodontal monitoring devices, thereby enabling combined presentation and analysis of oral health-related information beyond imaging data alone.

In some embodiments, the intraoral camera and AI-based analysis system are configured to comply with applicable FDA Class I or Class II regulatory requirements. The system may undergo periodic validation using reference intraoral imaging benchmarks to support continued conformity with evolving regulatory and quality standards. Regulatory classifications are referenced for illustrative purposes only and may vary based on jurisdiction, implementation, or regulatory updates.

In addition to FDA and FCC compliance, the camera system adheres to HIPAA and GDPR data protection protocols, ensuring that all patient health information (PHI) remains secure and encrypted. The system employs end-to-end AES-256 encryption for stored images and TLS 1.3 encryption for in-transit data, preventing unauthorized access.

In some embodiments, the systems and methods described herein are configured to comply with applicable regulatory, safety, and data protection standards for medical imaging and patient information handling. By way of example and without limitation, the system may satisfy United States Food and Drug Administration (FDA) Class I medical device requirements for dental and medical imaging applications, Federal Communications Commission (FCC) electromagnetic emission and safety compliance standards, and Bureau of Certification and Testing Center (BCTC) certification protocols validating product performance under standardized testing conditions.

In further embodiments, the system implements privacy and data protection controls consistent with Health Insurance Portability and Accountability Act (HIPAA) and General Data Protection Regulation (GDPR) requirements. Such controls may include end-to-end encryption, data anonymization or pseudonymization, access logging, and role-based access management to ensure secure handling of patient information across capture, storage, transmission, and analysis stages.

These certifications may position the systems and methods described herein as a trusted medical device for both home and professional use.

In some embodiments, the system integrates with Fast Healthcare Interoperability Resources (FHIR) and Health Level Seven (HL7) standards to facilitate seamless data sharing between dental professionals, healthcare providers, and electronic health record (EHR) platforms. This regulatory alignment ensures that AI-generated diagnostic insights can be securely incorporated into a patient's broader medical record, improving interdisciplinary collaboration in oral-systemic health care.

Some embodiments may include an AI-driven image processing system configured to analyze dental images in real-time while operating on a user's mobile device. Unlike conventional systems that may rely on cloud-based or remote server processing, the AI functionality in these embodiments may be embedded within the mobile application, which could help improve user privacy, reduce response times, and lower dependency on internet connectivity. The AI architecture may include a multi-layered filtering mechanism that identifies and removes non-diagnostic images, classifies anatomical features, and highlights areas of interest such as cavities, plaque accumulation, gum recession, or inflammation. Additionally, the system may facilitate tracking changes in dental conditions over time, allowing users and dental professionals to review trends and consider potential interventions.

In some embodiments, the system may be implemented using a modular framework configured to support future functional enhancements and specialized attachment components. By way of example and without limitation, interchangeable lens attachments may be provided in future versions, including fluorescence-based decay detection lenses configured to enable early identification of enamel demineralization and carious lesions.

In some implementations, the system may further support AI-enhanced periodontal health monitoring modules configured to evaluate gingival inflammation levels, gum recession rates, pocket depth indicators, and bacterial activity patterns derived from intraoral image data.

In further embodiments, hyperspectral imaging modules may be incorporated to perform wavelength-specific tissue analysis, thereby enabling early detection of oral cancer lesions, vascular abnormalities, and deep-tissue pathological conditions not readily visible using conventional imaging techniques.

In some implementations, telehealth and remote consultation features may be added, including real-time video streaming and bidirectional communication capabilities that allow dental professionals to remotely guide patients through self-conducted oral scanning procedures.

In some embodiments, the modular framework enables backward compatibility, standardized mechanical and electrical interfaces, and software extensibility, thereby allowing continuous expansion of imaging, analysis, monitoring, and telehealth capabilities without replacement of a core imaging device.

By adopting a modular approach, the systems and methods described herein may help ensure scalability, allowing new imaging technologies and AI capabilities to be integrated into future software and hardware updates.

Unlike traditional intraoral cameras with fixed lenses, the modular system allows hardware upgrades without replacing the entire device, ensuring long-term usability.

In some embodiments, the camera system may be configured to operate with a mobile application that provides AI-based capabilities for identifying and tracking a user's oral health conditions. The system may generate live AI-derived insights and highlight detected areas of concern to supplement professional dental evaluations.

In some embodiments, the mobile application provides a structured, guided user workflow that directs users through each stage of image acquisition, analysis, and clinical engagement. The workflow may begin with user authentication and profile creation, enabling individualized data association, personalized tracking, and multi-user account support within a same device environment.

In some implementations, the application performs secure camera connection and wireless configuration by pairing the intraoral camera with a mobile device through encrypted communication protocols, thereby enabling secure and reliable data transfer.

In further embodiments, the application provides real-time scan guidance using AI-driven pre-scan instructions that assist users in achieving optimal camera positioning, lighting, angle, and coverage. When additional guidance may be required, an integrated conversational interface or chat assistant may provide step-by-step instructions for camera operation, scanning technique, and application navigation.

In some embodiments, live AI analysis may be performed during image acquisition, with pathology detection algorithms processing images in real time to highlight potential areas of concern and generate immediate visual and textual feedback.

In further implementations, the application provides risk assessment and longitudinal trend tracking, enabling users to review pathology progression over time and receive automated alerts when detected conditions worsen or exceed predefined thresholds.

In some embodiments, the workflow further includes telehealth integration and appointment management features, allowing users to securely transmit scan data to dental professionals for remote evaluation and to schedule in-person or virtual consultations directly through the application interface.

In some implementations, the guided workflow integrates analysis, educational, and engagement features to reduce user error, improve scan quality, and promote consistent preventative oral health monitoring.

By structuring the user experience in this manner, the systems and methods described herein may improve user engagement, usability, and image analysis efficiency.

In some embodiments, the system provides technical and functional improvements relative to conventional intraoral cameras by enhancing image analysis reliability, usability, and user engagement. Unlike traditional intraoral cameras that primarily capture images for later review, the disclosed system integrates real-time AI-based analysis to provide immediate feedback regarding oral health condition indicators.

The Watch-It monitoring feature represents an example embodiment that may enable users to track specific areas of concern over time. Unlike prior solutions that require manual image comparison, Watch-It leverages AI-based tracking to detect progressive changes in dental condition indicators, such as cavity expansion, gum recession, and tooth surface degradation. The system alerts users if a monitored condition worsens, prompting earlier professional intervention.

The modularity of the system allows for future upgrades, including attachment-based enhancements such as specialized fluorescence-based decay detection, hyperspectral imaging for early-stage cavities, and periodontal imaging adapters. This modular approach ensures that the camera system remains adaptable to emerging dental technologies, differentiating it from fixed-function competitors.

### Operation of the Mobile Application

The mobile application may be any software capable of operating on any smart device that may be capable of a wired or wireless connection to the camera including a home computer, mobile phone or tablet. However, with reference to FIG. 11, a preferred mobile application may be an application (App) that incorporates AI capability and runs on a smartphone. The App allows the user to create and manage multiple profiles for the whole family. Each scan may be linked to a profile, enabling the application to track the progression of the oral health of everyone in the family. Its results page associates each concern with the tooth number where the pathology was detected. It also delineates the location of the pathology on the image to aid the user in comprehending the area of concern. Scanned images are stored in the Cloud for the AI to be re-trained and achieve better performance. The application also features the “Watch-It” functionality, which gives the user the ability to monitor a particular concern over time and be alerted if the issue may be deteriorating. For everyone with a profile on the App, it can be seen as a personal assistant who will keep an eye on their individual oral concern. The App provides a personal “Picture and Archiving System” (PACS) for the family to monitor their oral health over time. In addition, the Watch-It functionality may be an example AI feature that tracks and assesses the health of each user with a profile on an application implementing the systems and methods described herein.

In some embodiments, the disclosed system provides technical and functional advantages over conventional intraoral camera systems by integrating real-time artificial intelligence (AI) diagnostics, longitudinal scan history tracking, and preventative care analytics. Unlike imaging systems that merely capture and store images, the disclosed system performs real-time AI analysis to automatically identify dental and periodontal indicators, including cavities, plaque accumulation, gingival recession, and inflammation, without requiring immediate manual clinician review.

In some implementations, the system includes a longitudinal condition monitoring module configured to track progression of detected oral conditions across multiple scan sessions and to generate automated alerts when worsening trends, threshold exceedances, or abnormal progression patterns are identified.

In further embodiments, the system supports both local and cloud-based AI processing, enabling on-device analysis for real-time diagnostic feedback while synchronizing data with cloud storage for long-term historical tracking, population-based model refinement, and cross-session comparison.

In some embodiments, the system further supports a modular upgrade architecture in which interchangeable camera heads and attachment modules expand diagnostic functionality beyond standard intraoral imaging, thereby enabling additional imaging modalities and future diagnostic enhancements without replacement of a core imaging device.

Collectively, these features enable a unified diagnostic, monitoring, and preventative care platform that extends beyond conventional image capture workflows.

In some embodiments, the disclosed system may be configured to emphasize preventative care management and systemic health correlation in addition to intraoral imaging and tele-dentistry functionality.

In some implementations, the system provides a preventative-focused monitoring framework that integrates AI-based oral health analysis with systemic health correlation models. Rather than limiting functionality to remote consultation workflows, the system enables users to receive real-time diagnostic feedback, longitudinal condition tracking, and educational health insights that support proactive dental and medical care. The system further supports modular hardware expansion, multi-layered AI analysis, and cross-domain health correlation to extend diagnostic depth beyond conventional intraoral imaging systems.

In some embodiments, the system performs real-time AI analysis of intraoral scan data without requiring manual image review by a clinician prior to generating preliminary diagnostic indicators. The AI processing may be executed locally on a user device to provide immediate feedback while reducing reliance on remote cloud-based computation. This architecture supports preventative care engagement, longitudinal monitoring, and integrated oral-systemic health assessment.

In some embodiments, the disclosed systems and methods are distinguishable from existing orthodontic tracking and virtual consultation platforms by emphasizing preventative care, patient education, and integrated diagnostic intelligence. Rather than focusing primarily on remote consultation workflows, the disclosed system prioritizes early detection, longitudinal monitoring, and proactive oral health management.

In some implementations, the system may be configured to provide enhanced patient education by enabling users to visualize, understand, and track oral health conditions at early stages, thereby promoting informed decision-making and earlier intervention.

In further embodiments, the system performs live AI processing directly on a local device, enabling real-time diagnostic feedback while reducing reliance on cloud-based analysis and improving privacy, latency, and operational resilience.

In some embodiments, the system further incorporates systemic health tracking functionality in which oral health indicators are correlated with broader medical risk models to provide integrated health insights beyond dental-only assessment.

In addition, the system supports a modular camera head architecture that enables future expansion to additional imaging technologies, including fluorescence-based decay detection and hyperspectral imaging, thereby providing an upgrade pathway that extends beyond conventional intraoral imaging systems.

Unlike competitors that primarily serve orthodontists and tele-dentistry platforms, the systems and methods described herein may empower individual users with real-time AI analysis, enabling self-monitoring and early intervention for dental and systemic health concerns.

A differentiation may be that the system does not require constant internet connectivity for AI-powered insights. While some competitor platforms rely on remote processing and delayed analysis, some example embodiments leverage on-device AI models optimized for mobile hardware, allowing instant pathology detection without requiring data transmission to external servers. This approach enhances user privacy, speeds up diagnostic feedback, and ensures functionality in low-connectivity environments.

The modularity of the present system also distinguishes it from fixed-lens intraoral cameras. The ability to swap out imaging heads for fluorescence-based decay detection, hyperspectral imaging, and periodontal tissue assessment ensures long-term adaptability to emerging dental technologies. This future-proof design enables users to upgrade their diagnostic capabilities without replacing the entire device, providing a significant advantage over existing intraoral imaging solutions.

Some embodiments integrate insurance-linked scanning incentives, where patients who consistently use the device receive reduced out-of-pocket dental costs or eligibility for enhanced insurance coverage.

In some embodiments, the system integrates an AI-based analysis engine, real-time pathology tracking, and systemic health correlation features to provide a comprehensive intraoral imaging and monitoring platform suitable for both home and clinical environments.

Some embodiments may incorporate an intraoral camera that may be designed for home use, with a lightweight and ergonomic form factor. The optical system may include a high-precision lens assembly featuring a 0.6 mm aperture ring, which could extend the depth of field up to approximately 15 mm, with a sharp focus range between 5 mm and 20 mm. This design may help users capture high-quality images without requiring precise positioning.

To address potential image clarity challenges, such as condensation from breath, certain implementations may include a preheating system with precision-controlled resistors. In some example embodiments, this system may maintain the lens at an optimal temperature, such as around 94° F., to reduce condensation and improve imaging consistency. Unlike some existing solutions that may use passive anti-fog coatings or rely solely on LED-generated heat, an active heating mechanism could enable rapid temperature adjustments in response to environmental conditions.

The Watch-It feature monitors concerns of a specific tooth, and/or overall pathology for the mouth, e.g., including, in some cases, multiple teeth. Whenever a new scan may be completed, the App software models identify the tooth where the issue was initially found. Then, a segmentation model finds, rescales, and compares the issue now vs when it was first identified and estimates relative change over time. In some implementations, a “Watch-It” functionality may be included, enabling the tracking of individual teeth across successive scans to identify subtle changes. The AI models may be periodically updated through cloud-based learning, but on-device processing may provide users with immediate feedback without requiring continuous network connectivity.

Some embodiments may include functionality for tracking changes in tooth coloration over time. The system may analyze image data from successive scans to detect gradual shifts in tooth brightness and discoloration. This may allow users to monitor the effectiveness of whitening treatments or identify potential enamel degradation associated with dietary habits, smoking, or other factors.

In some embodiments, the mobile application implements an AI-driven workflow configured to streamline tele-dentistry services and insurance-linked preventative care management. The workflow may include AI-driven pre-scan instructions that guide a user regarding camera positioning, lighting conditions, and coverage regions to optimize image capture quality.

In further embodiments, the application performs real-time scan analysis during image acquisition, automatically highlighting detected areas of concern and flagging images that meet predefined criteria for professional review.

In some implementations, the system generates automated pathology reports that include AI-derived severity scores, confidence indicators, and progression metrics. The severity scoring framework may be calibrated using dentist-validated training data to support early clinical intervention.

In some embodiments, the workflow further includes tele-dentistry appointment booking functionality, enabling users to seamlessly schedule virtual consultations with licensed dental professionals directly through the application interface.

In further implementations, the application maintains insurance compliance tracking records by logging scan frequency, adherence metrics, and pathology trend indicators. The compliance records may be used to determine user eligibility for preventative care incentives, reimbursement benefits, or premium adjustments under value-based dental insurance programs.

In some embodiments, the AI-driven workflow integrates diagnostic, clinical, and insurance qualification functions into a unified preventative care management platform.

In some embodiments, the system integrates with insurance provider networks to reward users for consistent at-home monitoring. If an individual maintains a recommended scanning frequency and oral health condition, they may be eligible for discounted premiums or expanded dental coverage.

Unlike prior systems that only support image capture, this incentive-based AI model actively engages users in preventative care while enhancing telehealth accessibility.

Some embodiments may include an AI-based model configured to assess and compare tooth staining across multiple scans. The system may highlight areas where staining may be increasing or decreasing, allowing users to track the impact of lifestyle changes, oral hygiene habits, or dental treatments designed to reduce surface discoloration.

Some embodiments may be configured to identify and monitor wear patterns on teeth that may be indicative of grinding (bruxism). By analyzing changes in tooth shape, enamel wear, and microfractures, the system may provide insights into potential long-term effects and assist users in seeking preventative or corrective treatment.

In certain embodiments, the system may detect and track the progression of minor cracks or fractures in teeth. By comparing high-resolution images over multiple scans, the system may help users and dental professionals monitor the stability of existing cracks and assess whether they are worsening, potentially guiding decisions on restorative treatments.

In some embodiments, upon launching the mobile application, a user may be required to complete an authentication process before accessing system functionality. Following successful authentication, the user may create, edit, or delete one or more user profiles to enable personalized oral health tracking. The authenticated user may further initiate intraoral scans and view corresponding scan results, search for nearby dental professionals, and access historical scan records for review of prior findings and longitudinal comparisons.

In some implementations, the application transmits captured intraoral images to a cloud-based storage system for secure backup purposes. The cloud-stored image data may further be used, in accordance with applicable privacy and consent controls, to retrain and improve the AI models associated with the intraoral camera system, thereby enabling continuous enhancement of image analysis accuracy, segmentation performance, and pathology indicator detection capability over time.

Some embodiments may facilitate wireless data transmission between the intraoral camera and a mobile device through direct Wi-Fi and Bluetooth connections. A peer-to-peer Wi-Fi module may allow for direct communication between the devices, potentially supporting real-time image transmission even in environments where internet access is unavailable. This may reduce latency and allow AI processing to take place on the mobile device without requiring cloud-based computation.

Certain embodiments may also include an option for secure cloud storage, allowing users to retain image records for long-term tracking or to share scans with dental professionals. A cloud-based storage option may provide encryption protocols compliant with data privacy standards, enabling secure access for remote consultations. In some implementations, users may have the flexibility to choose between local AI processing and cloud-based review, depending on their needs.

Some implementations may integrate with dental insurance plans or discount programs to encourage regular at-home scanning. For example, the system may allow users to log scan frequency and quality, generating data that insurance providers could use to assess dental health trends. In some cases, users who meet certain scanning frequency or health maintenance criteria may qualify for incentives such as premium discounts or expanded preventative care coverage.

Unlike some prior systems that primarily support image capture, these embodiments may provide an incentive-based model to increase user engagement and promote proactive dental care. The integration of home-based monitoring with financial benefits may offer a way to improve compliance with recommended dental checkups and preventative care routines.

Some embodiments may be designed with modularity in mind, allowing for potential future enhancements. Additional features that may be incorporated into later versions could include specialized attachments for periodontal imaging, fluorescence-based decay detection, or energy-efficient models suited for areas with limited power availability.

Certain implementations may also support cross-platform compatibility, enabling expansion to tablet-based diagnostics, telehealth consultations, or AI-assisted care planning. As technology advances, the system may integrate with emerging diagnostic tools or be adapted for use in broader medical applications beyond dental monitoring.

The camera system may be offered as a subsidized device through insurance providers and dental discount programs. This business model reduces the upfront cost of the device and encourages widespread adoption of AI-powered preventative care. \

In some embodiments, the systems and methods described herein support a subscription-based AI analytics access model that enables users to selectively access enhanced diagnostic, tracking, and clinical service features. The subscription model may include multiple service tiers corresponding to different functional capabilities and user roles.

In some implementations, a basic service tier provides standard AI-assisted oral health monitoring, limited analytics features, and cloud-based image storage for longitudinal review.

In further embodiments, a premium service tier provides advanced pathology detection, systemic health correlation insights, predictive risk analytics, and expanded reporting capabilities derived from longitudinal scan data.

In some embodiments, a professional service tier, intended for clinical and institutional users, provides secure electronic health record (EHR) integration, automated patient report generation, and tele-dentistry consultation support features.

In some implementations, access permissions, data visibility, and feature availability are dynamically controlled based on subscription tier, thereby enabling scalable deployment across consumer, professional, and enterprise healthcare environments.

In some embodiments, the systems and methods described herein are configured to integrate with value-based dental insurance plans and employer wellness platforms to provide incentive-based preventative care programs. Users who maintain consistent oral health monitoring through regular intraoral scanning may qualify for discounted insurance premiums, expanded coverage for preventative dental treatments, and participation in employer wellness programs that incorporate AI-driven oral health tracking metrics.

In some implementations, the system generates compliance indicators, longitudinal oral health scores, and anonymized participation metrics that may be securely transmitted to participating insurance or wellness platforms in accordance with privacy and regulatory requirements. The incentive benefits may be dynamically adjusted based on continued scan adherence and oral health improvement trajectories, thereby reinforcing preventative care engagement.

In some embodiments, the business and deployment models described herein are configured to support preventative healthcare participation, early condition detection, and scalable user accessibility.

In some embodiments, the system supports insurance-linked and employer-sponsored deployment models that promote preventative oral healthcare adoption. For example, the intraoral camera device may be bundled with preventative care insurance plans, enabling users to obtain the device at a reduced cost upon enrollment in designated dental coverage programs.

In some implementations, advanced artificial intelligence (AI) analytics features are provided through a subscription-based access model in which users may select from a plurality of service tiers corresponding to different levels of diagnostic analysis, longitudinal tracking, and reporting functionality.

In further embodiments, the system may be deployed within employer-sponsored wellness programs in which the intraoral camera and associated application are offered as part of employee healthcare benefits. The wellness programs may incorporate AI-driven oral health monitoring metrics as indicators of preventative health participation and overall wellness engagement.

In some implementations, these deployment models collectively support scalable distribution, preventative care incentives, and integration of oral health monitoring into broader healthcare and wellness ecosystems.

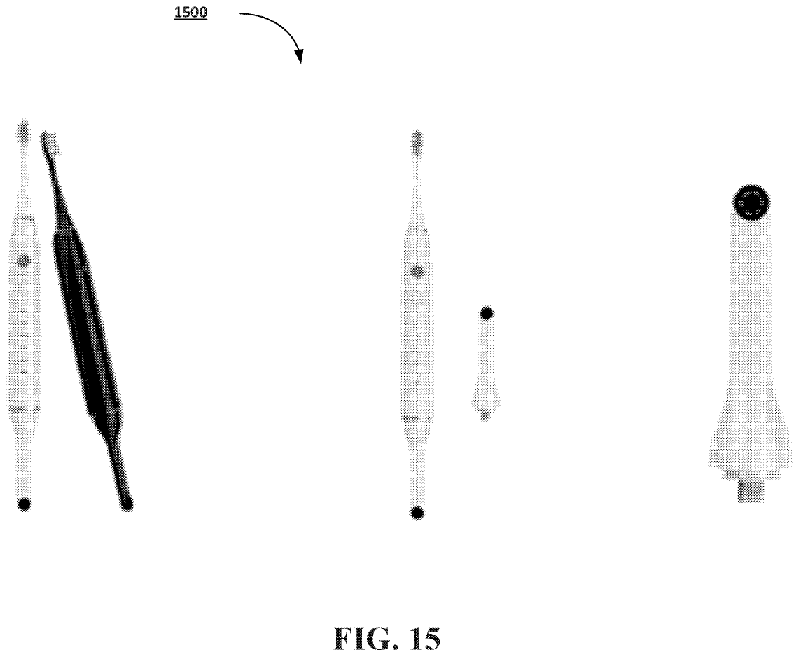

FIG. 15 is a diagram illustrating a device 1500 that may connect to an electric toothbrush and/or an intraoral camera. The intraoral camera, which may be part of a modular device, may be configured to establish a direct wireless connection with the mobile application installed on a smart device, such as a smartphone or tablet. In one embodiment, this connection may be made using a peer-to-peer Wi-Fi Direct link, which allows the device to communicate directly with the smart device without requiring a separate router or internet access. This configuration enhances portability and ensures the system can be used in home environments, remote locations, or areas where Wi-Fi infrastructure is unavailable.

In addition to functioning as an intraoral imaging device, the device shown in FIG. 15 may optionally include a detachable toothbrush head. In this configuration, the device may be operable in both an imaging mode and a brushing mode. When the toothbrush head may be attached, the same wireless connection may be used to transmit brushing-related data, including at least one of brush orientation, coverage region, motion pattern, or brushing duration. In some embodiments, the system may correlate the brushing-related data with previously captured intraoral image data to generate personalized brushing guidance within the mobile application.

The Wi-Fi connection supports real-time transmission of high-resolution video and still images from the camera to the mobile application. This direct wireless link allows the application to perform immediate AI analysis, image enhancement, and pathology detection while the scan may be occurring. The direct connection also reduces latency and ensures the video stream may be not degraded by network congestion, which could occur if images were first uploaded to a cloud server for processing. For brushing feedback, the system may overlay augmented reality (AR) guidance on the live feed, visually identifying areas requiring more attention.

In some embodiments, the device may further include Bluetooth functionality configured to assist with initial pairing and setup between the device and a smart device. After the initial setup, the system may automatically reconnect via a peer-to-peer Wi-Fi connection when the application is launched and the device is powered on, thereby facilitating repeat use for imaging, hygiene guidance, or other supported operations.

This wireless architecture supports both local image processing on the smart device and optional cloud-based storage or analysis, providing users with flexibility based on privacy preferences, network availability, and data management considerations. By enabling peer-to-peer Wi-Fi streaming, the system allows the device and mobile application to operate as a self-contained oral imaging and hygiene platform suitable for both clinical and home environments. The modular configuration of the device, which permits rapid interchange between a camera head and a toothbrush head, enhances versatility and encourages regular use for oral monitoring and daily hygiene support.

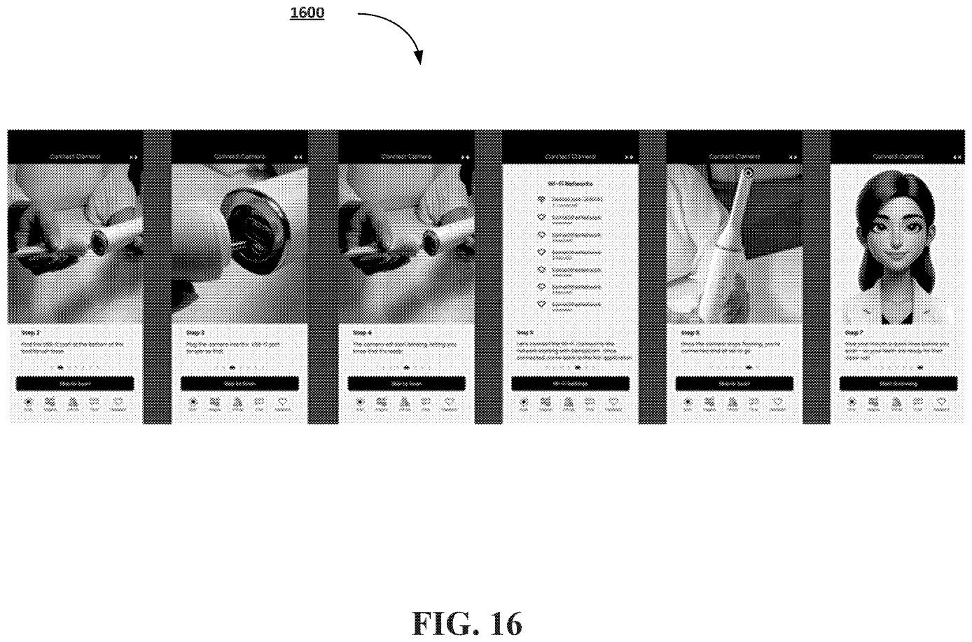

FIG. 16 is a diagram 1600 that illustrates how the intraoral camera device connects to the mobile application, enabling seamless data transmission between the hardware and software components of the system. In some embodiments, this connection may be established via Wi-Fi, ensuring the captured images and real-time video streams are transmitted directly to the user's smartphone or tablet. The figure highlights the importance of this wireless communication, which enables the system to perform real-time AI analysis, display live video feeds, and guide users through proper scanning techniques. This wireless link also supports the storage of captured images in the cloud and allows remote access by dental professionals when tele-dentistry features are enabled. The ability to pair the camera and app creates an integrated home monitoring system that combines hardware imaging capabilities with software-based AI processing and user feedback.

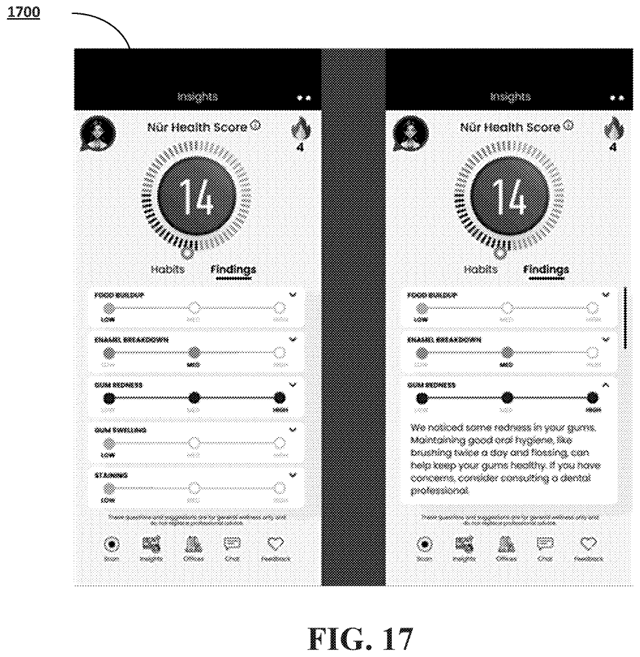

FIG. 17 provides an example user interface (UI) 1700 illustrating how the system's onboard AI engine identifies, ranks, and explains detected oral pathologies. This visual display helps users understand the specific dental conditions identified during a scan, such as dry mouth, gum redness, food buildup, evidence of tooth grinding, and enamel breakdown. In some embodiments, each detected issue may be assigned a severity level (e.g., Low, Medium, High), along with a brief description to educate the user on what the finding means and why it matters for their oral health. This AI-powered pathology detection and ranking ensures that users receive personalized insights into their oral health in a way that may be easy to understand, empowering them to take corrective action between dental visits. This transparency in AI findings may be a core feature that enhances user trust and supports preventative care

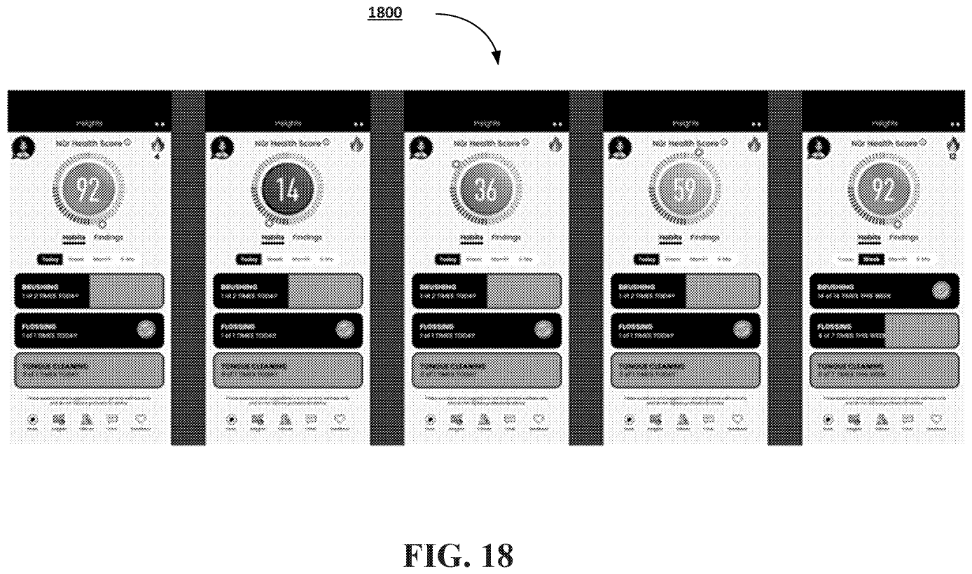

FIG. 18 illustrates an example habit-tracking interface 1800 provided within the mobile application. The habit tracker enables users to log and monitor daily oral care activities, including brushing, flossing, and tongue cleaning. The tracked activity data may be stored and analyzed over time to identify usage patterns and consistency trends. In some embodiments, the habit tracker may be associated with a health score metric such that consistent hygiene behavior contributes to an overall oral health assessment. The habit tracker may further include reminder notifications, streak counters, and adherence indicators to promote regular participation. By combining self-reported habit data with AI-derived scan results, the system generates a composite behavioral and clinical oral health profile for each user.

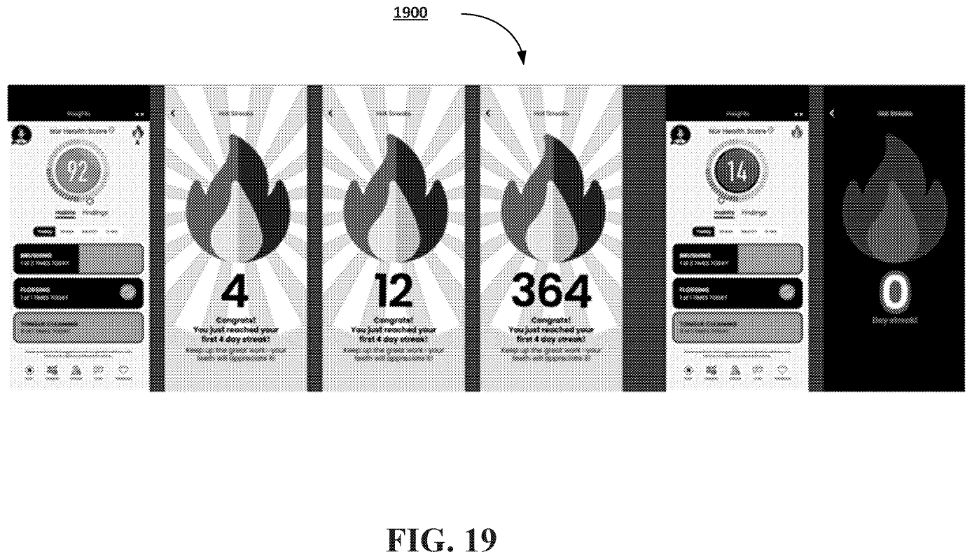

FIG. 19 illustrates an example user interface 1900 displaying a Health Score in a central graphical region together with a streak tracker indicating consecutive days of completed oral care activities. The displayed score may be dynamically updated based on survey responses, daily check-in data, and AI-derived scan findings. The streak tracker records consecutive days of user participation in designated hygiene and monitoring activities. In some embodiments, the combined score and streak indicators provide visual feedback reflecting longitudinal oral health behavior and monitoring consistency.

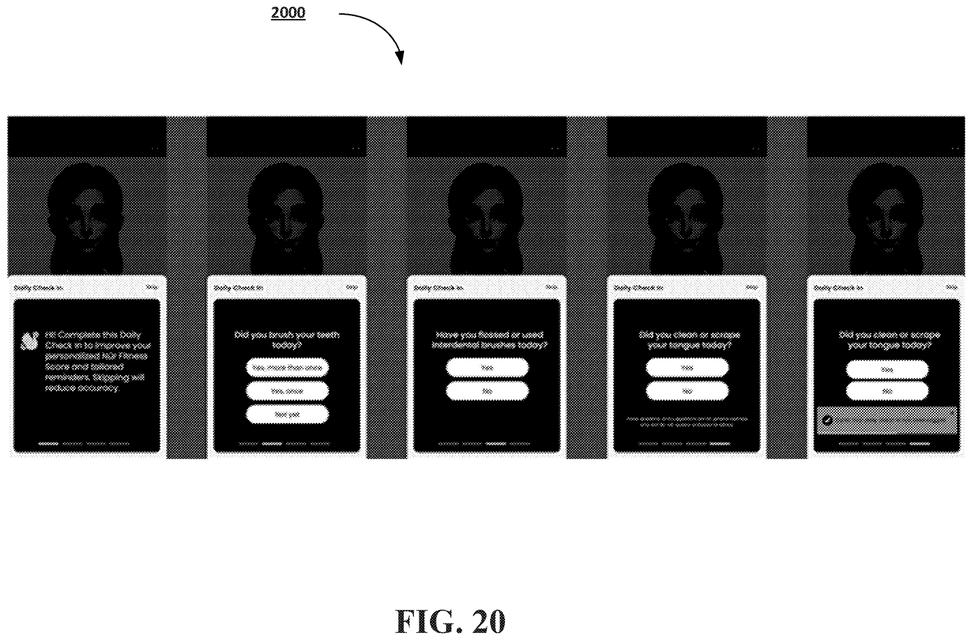

FIG. 20 illustrates an example daily check-in interface 2000 presenting a limited set of questions relating to daily oral hygiene behaviors, including brushing, flossing, and tongue cleaning. User responses are converted into a Daily Check-In Score that contributes to an overall health score metric. The simplified interface enables rapid user input while supporting consistent longitudinal data collection for behavioral trend analysis.

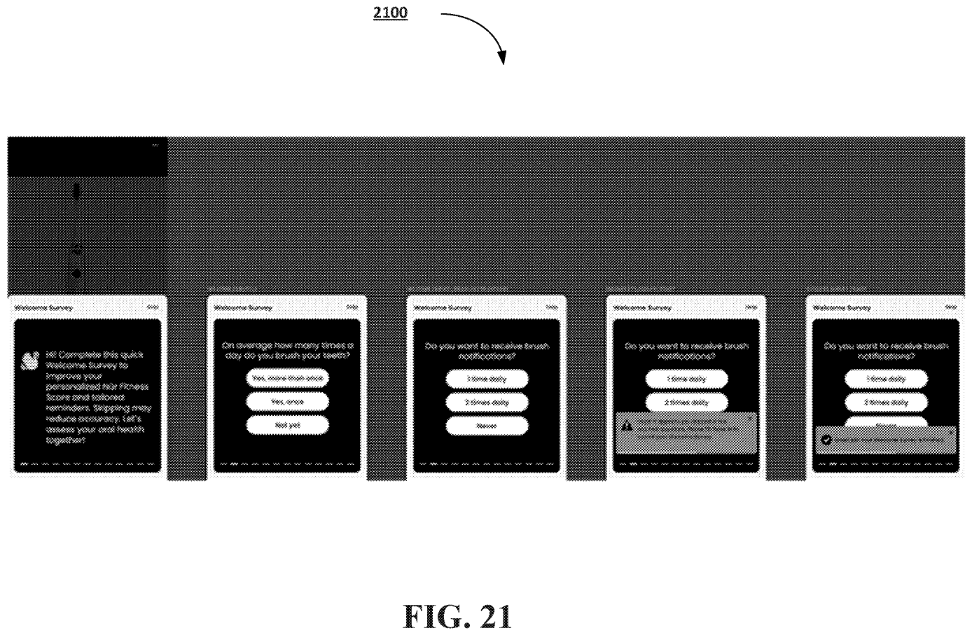

FIG. 21 illustrates an example Welcome Survey interface 2100 presented during initial profile creation. The Welcome Survey collects baseline information relating to oral hygiene habits, oral symptoms, lifestyle factors, and family or medical history associated with oral health. Each response may be assigned a numerical value contributing to an initial Welcome Survey Score. The resulting score establishes a baseline reference against which subsequent daily check-ins and AI-derived findings may be compared, thereby enabling detection of longitudinal trends, emerging risk indicators, and personalized assessment adjustments.

The following tables illustrate example health score calculations. The illustrated weightings, categories, scoring thresholds, and formulas are provided for explanatory purposes only. Other embodiments may use different scoring models, weight distributions, normalization techniques, category definitions, or threshold values.

### Example Health Score Calculation

TABLE 1A

| | | | Best Possible | Normalization | Final Weighted |
|---|---|---|---|---|---|
| Category | Weight | User Score | Score | Formula (Weight) | Score |
| Welcome | 40 | [User Total]/ | 30/30 | (User Total/30) × | [Final Score out |
| Survey | | 30 | | 40 | of 40] |
| Daily | 30 | [User Total]/ | 4/4 | (User Total/4) × | [Final Score out |
| Check-In | | 4 | | 30 | of 30] |
| AI | 30 | 30 − [User | 30 − 0 | 30 − (Penalty Sum) | [Final Score out |
| Findings | | Penalty] | | | of 30] |
| Total | 100 | Sum of all | 100 | (Welcome + | Final Score/100 |
| Overall | | categories | | Daily + AI Score) | |
| Score | | | | | |

TABLE 1B

| | | | Best Possible | Normalization | Final Weighted |
|---|---|---|---|---|---|
| Category | Weight | User Score | Score | Formula (Weight) | Score |
| Welcome | 25 | [User Total]/ | 31/31 | (User Total/30) × | [Final Score out |
| Survey | | 31 | | 40 | of 40] |
| Daily | 30 | [User Total]/ | 4/4 | (User Total/4) × | [Final Score out |
| Check-In | | 4 | | 30 | of 30] |
| AI | 45 | 40 − [User | 40 − 0 | 40 − (Penalty Sum) | [Final Score out |
| Findings | | Penalty] | | | of 45] |
| Total | 100 | Sum of all | 100 | (Welcome + | Final Score/100 |
| Overall | | categories | | Daily + AI Score) | |
| Score | | | | | |

In some embodiments, generation of the health score requires the presence of scoring inputs from each of the defined scoring categories, including the Welcome Survey, the Daily Check-In, and AI Findings. The Welcome Survey may be completed once during initial user onboarding and establishes a baseline score that persists for subsequent calculations unless updated by the user.

For returning users, the system may dynamically update the health score during successive application sessions. Upon user login, the system determines whether a Daily Check-In has been completed and, when present, updates the health score based on reported oral hygiene behaviors associated with a preceding time period. In some implementations, completion of an intraoral scan triggers additional processing in which AI-derived findings are analyzed and incorporated into the health score calculation, resulting in a further update to the overall score.

Each scoring category may be processed independently such that updates to one category modify only the corresponding component score while maintaining previously calculated values for remaining categories. The overall health score may then be recalculated by aggregating the independently updated component scores, thereby enabling incremental and real-time adjustment of the user's health assessment as new behavioral or diagnostic information becomes available.

Tables 1A-1B outline examples of health score calculation, which combines self-reported data, user engagement, and AI-driven analysis to generate an overall health score. The system assigns weighted scores to three categories: for example, from TABLE 1A, the Welcome Survey (40 points), which evaluates the user's initial health status; the Daily Check-In (30 points), which tracks regular engagement with the system; and AI Findings (30 points), which starts at a perfect score of 30 but deducts points based on detected health issues. Each category may be normalized based on predefined maximum values to ensure consistency in scoring. The final overall score, ranging from 0 to 100, may be calculated as the sum of these weighted components, providing a comprehensive measure of a user's oral health and engagement with the monitoring system.

For TABLE 1B, the health score calculation may combine multiple categories reflecting baseline health characteristics, daily behavioral engagement, and AI-derived clinical findings. In some embodiments, the Welcome Survey (25 points) evaluates a user's initial oral health status and establishes a baseline assessment; the Daily Check-In (30 points) tracks ongoing user engagement and reported oral hygiene behaviors; and AI Findings (45 points) reflect objective analysis of intraoral scans, where the category begins from a maximum score and deductions may be applied based on detected oral health conditions. Each category may be normalized according to predefined maximum values and weighting factors to ensure consistent scoring across users and time periods. The final overall score, ranging from 0 to 100, may be calculated as an aggregation of the weighted category scores, thereby providing a composite indicator of both oral health condition and user adherence to recommended monitoring and hygiene practices.

In some example embodiments, the Welcome Survey may be a one-time assessment completed by users when they first set up their profile in the system. The Welcome Survey gathers baseline information about the user's oral health habits, potential risk factors, and overall dental history. The Welcome Survey may be reprompted to users after some time has passed to reassess their oral health status and update recommendations based on any changes in habits or conditions.

The Example Welcome Survey includes twelve multiple-choice questions covering key oral health topics. These topics include brushing and flossing frequency, symptoms such as gum sensitivity and dryness, lifestyle factors such as tobacco use and sugary drink consumption, and family history of dental issues.

Each response to the Welcome Survey may be assigned a numeric value. The total of all responses results in a raw Welcome Survey score, which may be normalized to a maximum of 40 points to fit within the overall Health Score Calculation. The normalization formula used is:

$$\frac{Total Welcome Survey Score}{30} \times 40$$

This may help ensure consistent weighting regardless of variations in user responses.

The following illustrates an example Welcome Survey as may be implemented in some embodiments:

### First Example Welcome Survey

Description: This may be a one-time welcome survey, for first time users.

Q1. “On average, how many times do you brush per day?”

### Possible Answers & Numeric Values

┘ 0=“I don't brush daily”
┘ 1=“Once a day”
   | 2=“Twice a day”
   | 3=“More than twice a day”
   Q2. “How often do you floss or clean between your teeth?”

### Possible Answers & Numeric Values

┐ 0=“Never”
┘ 1=“A few times a week”
┘ 2=“Daily”
┐ 3=“Multiple times daily”
   Q3. “Do you clean or scrape your tongue?”

### Possible Answers & Numeric Values

• 0=“Never”
• 1=“Rarely”
• 2=“Sometimes”
• 3=“Daily”
   Q4. “Have you noticed any gum sensitivity, bleeding, or soreness in the past few months?”

### Possible Answers & Numeric Values

┐ 0=“Often”
┘ 1=“Occasionally”
┘ 2=“Never”
   Q5. “Do you suspect or know that you grind/clench your teeth?”

### Possible Answers & Numeric Values

┘ 0=“Yes, frequently”
┐ 1=“Yes, occasionally”
   | 2=“Not sure”
⊐ 3=“No”
   Q6. “How often do you experience dryness or a sticky feeling in your mouth?”
□ 0=“Often”
□ 1=“Sometimes”
□ 2=“Rarely”
□ 3=“Never”
   Q7. “Are you taking any medications that can cause dry mouth (e.g., certain antihistamines, antidepressants)?”
□ 0=“Yes, I experience dryness often”
□ 1=“Yes, but mild dryness”
□ 2=“Not sure”
□ 3=“No”
   Q8. “Do you smoke or use tobacco products?”
∘ 0=“Yes”
∘ 1=“No”
   Q9. “How often do you consume sugary drinks (soda, juice, sports drinks)?”
∘ 0=“Daily or more”
∘ 1=“Often”
∘ 2=“Rarely”
∘ 3=“Never”
   Q10. “Are your teeth sensitive to hot or cold foods/drinks?”

### Possible Answers & Numeric Values

┐ 0=“Often”
ℏ 1=“Sometimes”
┘ 2=“Never”
   Q11. “Has anyone in your immediate family had significant gum disease or early tooth loss?”

### Possible Answers & Numeric Values

   | 0=“Yes, multiple family members”
⊐ 1=“Yes, at least one”
⊐ 2=“Not Sure”
┘ 3=“No”
   Q12. “Do you have any chronic health conditions (like diabetes) that might affect oral health?”

### Possible Answers & Numeric Values

   | 0=“Yes, not well controlled”
⊐ 1=“Yes, but controlled”
⊐ 2=“Not Sure”
┘ 3=“No known conditions”

Scoring: Max possible score: 30

Each of the 12 questions has values between 0-3 (except Q8 with 0-1).

Normalize to 40 points by using: (Total Score/30)×40

### Second Example Welcome Survey

### 1. Welcome Survey

Description: This is a one-time welcome survey, for first time users.
Q1. “On average, how many times do you brush per day?”

### Possible Answers & Numeric Values

⊐ 0=“I don't brush daily”
┐ 1=“Once a day”
┘ 2=“Twice a day”
⊐ 3=“More than twice a day”
   Q2. “How often do you floss or clean between your teeth?”

### Possible Answers & Numeric Values

   | 0=“Never”
┘ 1=“A few times a week”
┐ 2=“Daily”
⊐ 3=“Multiple times daily”
   Q3. “Do you clean or scrape your tongue?”

### Possible Answers & Numeric Values

• 0=“Never”
• 1=“Rarely”
• 2=“Sometimes”
• 3=“Daily”
   Q4. “Have you noticed any gum sensitivity, bleeding, or soreness in the past few months?”

### Possible Answers & Numeric Values

┐ 0=“Often”
⊐ 1=“Occasionally”
┐ 2=“Never”
   Q5. “Do you suspect or know that you grind/clench your teeth?”

### Possible Answers & Numeric Values

   | 0=“Yes, frequently”
┘ 1=“Yes, occasionally”
   | 2=“Not sure”
┐ 3=“No”
   Q6. “How often do you experience dryness or a sticky feeling in your mouth?”
□ 0=“Often”
□ 1=“Sometimes”
□ 2=“Rarely”
□ 3=“Never”
   Q7. “Are you taking any medications that can cause dry mouth (e.g., certain antihistamines, antidepressants)?”
□ 0=“Yes, I experience dryness often”
□ 1=“Yes, but mild dryness”
□ 2=“Not sure”
□ 3=“No”
   Q8. “Do you smoke or use tobacco products?”
∘ 0=“Yes”
∘ 1=“No”
   Q9. “How often do you consume sugary drinks (soda, juice, sports drinks)?”
∘ 0=“Daily or more”
∘ 1=“Often”
∘ 2=“Rarely”
∘ 3=“Never”
   Q10. “Are your teeth sensitive to hot or cold foods/drinks?”

### Possible Answers & Numeric Values

┘ 0=“Often”
⊐ 1=“Sometimes”
   | 2=“Never”
   Q11. “Has anyone in your immediate family had significant gum disease or early tooth loss?”

### Possible Answers & Numeric Values

   | 0=“Yes, multiple family members”
┐ 1=“Yes, at least one”
┐ 2=“Not Sure”
⊐ 3=“No”
   Q12. “Do you have any chronic health conditions (like diabetes) that might affect oral health?”

### Possible Answers & Numeric Values

┘ 0=“Yes, not well controlled”
   | 1=“Yes, but controlled”
┐ 2=“Not Sure”
┐ 3=“No known conditions”

Scoring: Max possible score: 31

Each of the 12 questions has values between 0-3 (except Q4,Q8,Q10 with 0-1 and 0-2).

$$Weighted score = ( Total Score / 31 ) \times 25 \%$$

### 2. Daily Check-In

Description: This is a daily check-in, that users are supposed to answer every day upon app start.
Q1. “Did you brush your teeth today?”

### Possible Answers & Numeric Values

┐ 0=“No/Not Yet”
┐ 1=“Yes, once”
⊐ 2=“Yes, more than once”
   Q2. “Have you flossed today?”

### Possible Answers & Numeric Values

⊐ 0=“No/Not Yet”
   | 1=“Yes, once”
┘ 2=“Yes, more than once”
   Q3. “Did you scrape or clean your tongue today?”

### Possible Answers & Numeric Values

┐ 0=“No/Not Yet”
   | 1=“Yes, once”
┐ 2=“Yes, more than once”
Scoring: Max Score=6
Brushing (0-2)
Flossing (0-2)
Tongue Cleaning (0-2)
Total Score Range: 0-6Weighted score=(Total Score/6)×30%

### AI Findings

Description: Findings are detected upon each scan and can change over time.
1. Gum redness (inflammation)
   a. Detected
   b. Not Detected
2. Gum recession
   a. Detected
   b. Not Detected
3. Calculus/Tartar
   a. Detected
   b. No Detected
4. Dental Caries/Decay
   a. Detected
   b. No Detected

### Scoring:

The 5 AI Findings are rated Detected or Not Detected. Assigned penalties:
⊐ Detected=−10
┐ Not Detected=0
   Sum all penalties (Max negative score: −40)

$$Weighted score = ( Total Score / 40 ) \times 45 \%$$

In some embodiments, the Welcome Survey provides the initial data set against which future data (including Daily Check-Ins and AI Findings) are compared. This allows the system to detect changes over time, identify emerging risks, and personalize oral health recommendations.

In some example embodiments, the Daily Check-In may be a brief assessment presented to users each day upon launching the mobile application. The Daily Check-In tracks ongoing oral hygiene habits by asking whether the user brushed, flossed, and cleaned their tongue that day.

Each question in the Daily Check-In may be assigned a numeric value reflecting the user's response. For example, brushing may be scored on a scale from 0 to 2, while flossing and tongue cleaning are each scored on a scale of 0 to 1. The responses are combined into a daily hygiene score with a maximum value of 4 points.

To ensure consistency in the Health Score Calculation, the daily hygiene score may be normalized to a 30-point scale using the formula:

$$\frac{Daily Check - In Score}{4} \times 30$$

This normalization allows the Daily Check-In to contribute proportionally to the overall health score, emphasizing the importance of regular oral hygiene practices.

In some embodiments, the system may track the user's Daily Check-In trends over time, identifying patterns of skipped hygiene routines or inconsistent behavior. This historical data may be factored into future health assessments, allowing the system to detect gradual declines in oral health habits even before pathological signs emerge:

### 2. Daily Check-In

Description: This may be a daily check-in, that users are supposed to answer every day upon app start.

Q1. “Did you brush your teeth today?”

### Possible Answers & Numeric Values

┐ 0=“No/Not Yet”
┘ 1=“Yes, once”
┘ 2=“Yes, more than once”
   Q2. “Have you flossed or used interdental brushes today?”

### Possible Answers & Numeric Values

⊐ 0=“No/Not Yet”
   | 1=“Yes”
   Q3. “Did you scrape or clean your tongue today?” Possible Answers & Numeric Values
⊐ 0=“No/Not Yet”
   | 1=“Yes”
Scoring: Max Score=4:
Brushing (0-2)
Flossing (0-1)
Tongue Cleaning (0-1)
Total Score Range: 0-4
Normalize to 30 points using: (Total Score/4)×30

In addition to user-reported data, some embodiments include AI Findings, which are automatically generated each time the user completes an intraoral scan. The AI Findings reflect real-time clinical observations detected by the camera system and image processing algorithms.

AI Findings may include indicators such as dry mouth, gum redness, food buildup, tooth grinding evidence, and enamel breakdown. Each finding may be assigned a severity level—Low, Medium, or High—based on the system's analysis of the scanned images.

To generate an objective AI Findings Score, the system applies penalties based on severity. Findings rated “Low” incur no penalty, while “Medium” findings result in a −2 penalty, and “High” findings result in a −4 penalty. The total AI penalty score may be then subtracted from the maximum AI score of 30.

The final AI Findings Score may be normalized to fit within the 30-point allocation of the Health Score Calculation using the formula:

$$30 + ( Total AI Penalty Score )$$

This structure ensures that users with consistently healthy scans receive the full 30 points, while users with persistent or worsening findings receive progressively lower scores.

In some embodiments, the system tracks longitudinal changes in the AI Findings to monitor the progression of identified conditions over time. This historical tracking allows the system to distinguish between stable, improving, or deteriorating conditions, enabling the system to issue early warnings or recommend more frequent professional consultation if necessary.

### AI Findings

Description: Findings are detected upon each scan and can change over time.
1. Dry Mouth (dry tongue/sticky saliva)
   a. Low
   b. Medium
   c. High
2. Gum redness
   a. Low
   b. Medium
   c. High
3. Food buildup
   a. Low
   b. Medium
   c. High
4. Tooth grinding
   a. Low
   b. Medium
   c. High
5. Enamel Breakdown
   a. Low
   b. Medium
   c. High

### Scoring:

The 5 AI Findings are rated Low, Medium, or High. Assigned penalties:
┘ Low=0
┐ Medium=−2
   | High=−4

Sum all penalties (Max negative score: −20):

Normalize by adding 30, so final score is: 30+(Total AI Penalty Score.) If no issues, user gets full 30 points.

### Oral Health Score Ranges (Example 1)

TABLE 2A

| Score | | |
|---|---|---|
| Range | Rating | Interpretation & Recommendations |
| 90-100 | Excellent | Your oral health habits are outstanding! Keep up the great work with |
| | | brushing, flossing, tongue cleaning, and overall care. Maintain regular |
| | | dental check-ups and continue your excellent routine. |
| 80-89 | Very Good | Your oral health is strong, but there's room for minor improvements. |
| | | Ensure you floss daily, clean your tongue regularly, and stay hydrated |
| | | to prevent dry mouth. Monitor AI findings for any early signs of |
| | | issues. |
| 70-79 | Good | You have a solid foundation, but consistency matters! Focus on |
| | | brushing twice daily, flossing more frequently, and addressing any AI- |
| | | detected concerns like gum redness or food buildup. |
| 60-69 | Moderate | Some habits need improvement. You might be missing flossing days, |
| | Risk | skipping tongue cleaning, or experiencing signs of sensitivity or gum |
| | | irritation. Adjust your routine and consider a dental check-up. |
| 40-59 | High Risk | There are noticeable concerns in your oral health routine. You may |
| | | have dry mouth, gum redness, or frequent issues with sensitivity. |
| | | Increase brushing, flossing, and hydration. Professional guidance is |
| | | recommended. |
| Below | Severe | Your oral health is at serious risk. There may be frequent dry mouth, |
| 40 | Risk | tooth grinding, or enamel breakdown. Immediate action is required- |
| | | see a dentist, improve your daily habits, and track AI-detected issues |
| | | closely. |

### Oral Health Score Ranges

TABLE 2B

| Score | | |
|---|---|---|
| Range | Rating | Interpretation & Recommendations |
| 90- | Excellent | Your oral health habits are outstanding! Keep up the great work |
| 100 | | with brushing, flossing, tongue cleaning, and overall care. Maintain |
| | | regular dental check-ups and continue your excellent routine to |
| | | help maintain your oral health. |
| 80-89 | Very Good | Your oral health is strong, but there's room for minor improvements. |
| | | Ensure you floss daily, clean your tongue regularly, stay hydrated |
| | | to prevent dry mouth, and implement recommendations to help |
| | | maintain oral health. |
| 70-79 | Good | You have a solid foundation, but consistency matters! Focus on |
| | | brushing twice daily, flossing more frequently, and implement |
| | | recommendations to help improve oral health. |
| 60-69 | Moderate | Some habits need improvement. You might be missing flossing |
| | Risk | days, skipping tongue cleaning, or experiencing signs of sensitivity |
| | | or gum irritation. Adjust your routine and consider a dental |
| | | check-up to help improve you oral health. |
| 40-59 | High Risk | There are noticeable concerns in your oral health routine. You may |
| | | have dry mouth, gum redness, or frequent issues with sensitivity. |
| | | Increase brushing, flossing, and hydration, and professional |
| | | guidance is recommended to help improve your oral health |
| Below | Severe | Your oral health is at serious risk. There may be frequent dry mouth, |
| 40 | Risk | tooth grinding, or enamel breakdown. Immediate action is required- |
| | | see a dentist, and, your daily habits to help improve your oral health. |

Tables 2A and 2B define examples of Oral Health Score Ranges, providing users with clear feedback based on their combined survey, daily check-in, and AI findings scores. Scores from 90 to 100 indicate Excellent oral health, encouraging users to maintain their strong habits, while scores below 40 signal a Severe Risk, requiring immediate attention and professional care. Each range includes personalized recommendations, such as increasing flossing frequency, addressing dry mouth, or scheduling a dental visit, helping users understand both their current status and the steps needed to improve. This tiered system provides actionable guidance, turning the Health Score into a practical tool for ongoing oral health management.

Table 3 provides a practical example of how the Health Score Calculation works by illustrating Sarah's responses to the Welcome Survey. In this example, Sarah's answers reflect fair oral health habits, with some areas for improvement, such as flossing frequency, tongue cleaning, and family history of gum disease. Sarah's total raw score from the survey is 20 out of 30 possible points. This score may be then normalized to a 40-point scale, resulting in a final Welcome Survey score of 26.67 points. This normalized score becomes part of Sarah's overall health score, demonstrating how initial self-reported data influences the system's assessment and future recommendations. This example highlights how the system translates individual responses into a quantifiable and trackable health metric.

### First Example: Sarah

Sarah's Welcome Survey Responses (Max Score=30, Normalized to 40)

TABLE 3

| | | | Best Case |
|---|---|---|---|
| Question | User Response | Score | Score |
| Q1 (Brushing) | “Twice a day” | 2 | 3 |
| Q2 (Flossing) | “A few times a week” | 1 | 3 |
| Q3 (Tongue Cleaning) | “Sometimes” | 2 | 3 |
| Q4 (Gum Sensitivity) | “Occasionally” | 1 | 2 |
| Q5 (Teeth Grinding) | “Yes, occasionally” | 1 | 3 |
| Q6 (Dry Mouth) | “Rarely” | 2 | 3 |
| Q7 (Medication) | “Not sure” | 2 | 3 |
| Q8 (Tobacco) | “No” | 1 | 1 |
| Q9 (Sugary Drinks) | “Rarely” | 2 | 3 |
| Q10 (Sensitivity) | “Never” | 2 | 2 |
| Q11 (Family Gum Disease) | “Yes, at least one” | 1 | 3 |
| Q12 (Chronic Condition) | “No known conditions” | 3 | 3 |

Total User Score: (20/30)
Normalized to 40: (20/30) × 40 = 26.67

Table 4 presents Sarah's Daily Check-In Responses, demonstrating how her self-reported daily oral care routine translates into her Daily Check-In score. Sarah indicated that she brushed her teeth more than once and flossed, earning full points for both activities. However, she did not clean her tongue, resulting in a zero for that category. Her total score for the check-in is 3 out of a possible 4 points, which may be then normalized to 30 points using the formula:

$$\frac{3}{4} \times 30 = 22.5$$

This normalized score contributes directly to Sarah's overall Health Score, reflecting her day-to-day commitment to essential oral hygiene habits. Table 4 highlights how simple daily actions—or the lack of them—impact Sarah's ongoing health assessment.

### Sarah's Daily Check-In Responses (Max Score=4, Normalized to 30)

TABLE 4

| Question | User Response | Score | Best Case Score |
|---|---|---|---|
| Brushed Today? | “Yes, more than once” | 2 | 2 |
| Flossed Today? | “Yes” | 1 | 1 |
| Tongue Cleaned Today? | “No/Not Yet” | 0 | 1 |

Total User Score: (3/4)
Normalized to 30: (3/4) × 30 = 22.5

Table 5 presents Sarah's AI Findings, which reflect the system's automated analysis of her intraoral scans. In this example, the AI detected Medium dry mouth, Medium food buildup, and High evidence of tooth grinding, resulting in penalties of −2, −2, and −4 points, respectively. The AI also detected Low gum redness and Low enamel breakdown, which incur no penalties.

The penalties are subtracted from the maximum AI score of 30, giving Sarah a final AI Findings score of 22 points. This penalty-based scoring system ensures that users with healthier scans maintain higher scores, while users with multiple or severe oral health issues see reductions in their AI Findings score. Combined with Sarah's Welcome Survey and Daily Check-In scores, this AI Findings score contributes to her overall Health Score, providing a complete, data-driven picture of Sarah's oral health condition.

### Sarah's AI Findings (Max Score=30, Penalty-Based)

TABLE 5

| Finding | AI Result | Penalty | Best Case (No Issues) |
|---|---|---|---|
| Dry Mouth | Medium | −2 | 0 |
| Gum Redness | Low | 0 | 0 |
| Food Buildup | Medium | −2 | 0 |
| Tooth Grinding | High | −4 | 0 |
| Enamel Breakdown | Low | 0 | 0 |

Total User Penalty = (−8)
Final AI Score: 30 − 8 = 22

Table 6 provides Sarah's Final Score, summarizing her performance across all three key categories: the Welcome Survey, the Daily Check-In, and the AI Findings. Sarah's Welcome Survey score of 26.67 out of 40 reflects her baseline oral health habits and risk factors. Her Daily Check-In score of 22.5 out of 30 captures her ongoing hygiene efforts, while her AI Findings score of 22 out of 30 reflects the system's objective assessment of her actual oral health condition based on scan analysis.

Together, these scores combine into a final Health Score of 71.17 out of 100, placing Sarah in the “Good” range. This indicates that Sarah has a solid foundation but also highlights areas for improvement, such as more consistent tongue cleaning and addressing her tooth grinding issues. By tracking these combined factors over time, the system can provide personalized recommendations and monitor her progress toward improved oral health.

### Sarah's Final Score

TABLE 6

| Category | User Score | Best Possible Score |
|---|---|---|
| Welcome Survey | 26.67/40 | 40/40 |
| Daily Check-In | 22.5/30 | 30/30 |
| AI Findings | 22/30 | 30/30 |
| Final Score | 71.17/100 | 100/100 |

71. Good, but some areas need improvement.
Strong areas: Regular brushing & flossing
Needs improvement: Tongue cleaning, reducing tooth grinding.

### Second Example: Sarah

### Sarah's Welcome Survey Responses (Max Score=30, Normalized to 40)

| | | | Best Case |
|---|---|---|---|
| Question | User Response | Score | Score |
| Q1 (Brushing) | “Twice a day” | 2 | 3 |
| Q2 (Flossing) | “A few times a week” | 1 | 3 |
| Q3 (Tongue Cleaning) | “Sometimes” | 2 | 3 |
| Q4 (Gum Sensitivity) | “Occasionally” | 1 | 2 |
| Q5 (Teeth Grinding) | “Yes, occasionally” | 1 | 3 |
| Q6 (Dry Mouth) | “Rarely” | 2 | 3 |
| Q7 (Medication) | “Not sure” | 2 | 3 |
| Q8 (Tobacco) | “No” | 1 | 1 |
| Q9 (Sugary Drinks) | “Rarely” | 2 | 3 |
| Q10 (Sensitivity) | “Never” | 2 | 2 |
| Q11 (Family Gum Disease) | “Yes, at least one” | 1 | 3 |
| Q12 (Chronic Condition) | “No known conditions” | 3 | 3 |

Total User Score: (20/30)
Normalized to 40: (20/30) × 40 = 26.67

### Sarah's Daily Check-In Responses (Max Score=4, Normalized to 30)

| Question | User Response | Score | Best Case Score |
|---|---|---|---|
| Brushed Today? | “Yes, more than once” | 2 | 2 |
| Flossed Today? | “Yes” | 1 | 1 |
| Tongue Cleaned Today? | “No/Not Yet” | 0 | 1 |

Total User Score: (3/4)
Normalized to 30: (3/4) × 30 = 22.5

### Sarah's AI Findings (Max Score=30, Penalty-Based)

| Finding | AI Result | Penalty | Best Case (No Issues) |
|---|---|---|---|
| Dry Mouth | Medium | −2 | 0 |
| Gum Redness | Low | 0 | 0 |
| Food Build-up | Medium | −2 | 0 |
| Tooth Grinding | High | −4 | 0 |
| Enamel Breakdown | Low | 0 | 0 |

Total User Penalty = (−8)
Final AI Score: 30 − 8 = 22

### Sarah's Final Score

| Category | User Score | Best Possible Score |
|---|---|---|
| Welcome Survey | 26.67/40 | 40/40 |
| Daily Check-In | 22.5/30 | 30/30 |
| AI Findings | 22/30 | 30/30 |
| Final Score | 71.17/100 | 100/100 |

71. Good, but some areas need improvement.
Strong areas: Regular brushing & flossing
Needs improvement: Tongue cleaning, reducing tooth grinding

### Discount Plan Model

In an additional aspect of the invention, the dental camera may be given to a consumer as a benefit of buying dental insurance. In a more preferred embodiment, the dental camera may be given away, or sold at a discount, to a person purchasing a dental discount plan for themselves or for their family. Preferably, the dental discount plan offers significant discounts on most dental procedures (such as a 20% to 50% savings). In some implementations, the plan may not constitute traditional insurance coverage, accordingly, in some examples, there are no deductibles or annual maximums. In addition to the insurance plan and/or discount plan, the plan provides patients with real-time visibility into their treatment savings and costs-all on their phone.

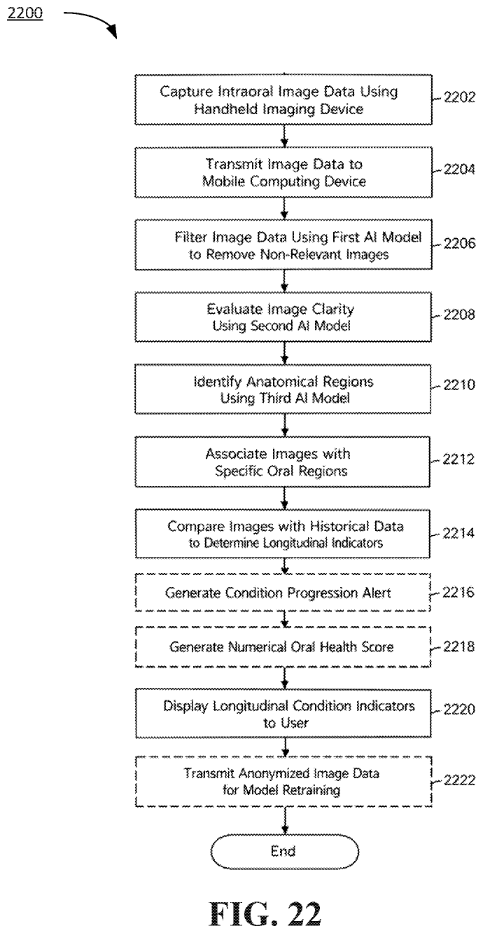

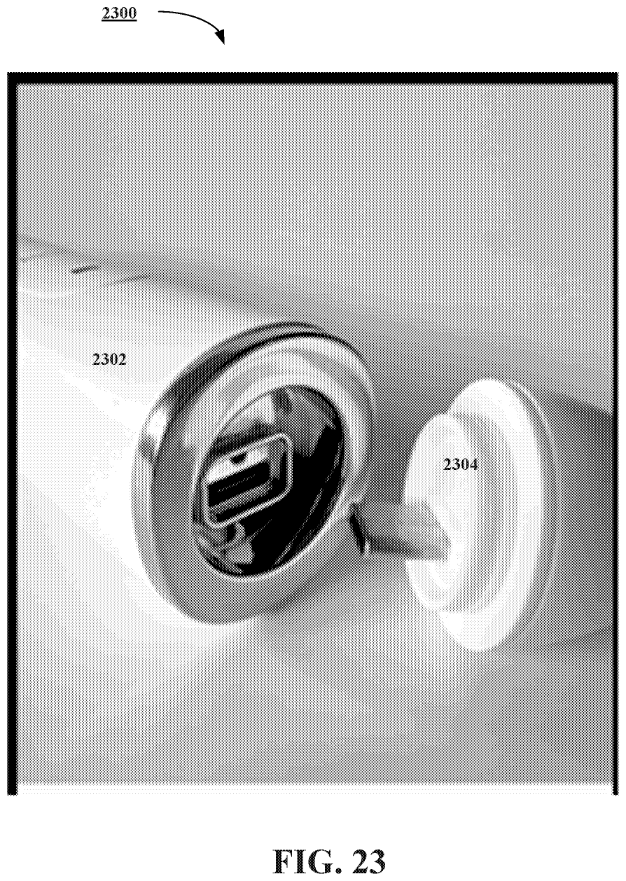

FIG. 22 illustrates a flowchart of an example method 2200 for monitoring oral health conditions, including one or more of the following steps: capturing intraoral image data (2202), transmitting the image data to a mobile computing device (2204), filtering the image data using a first artificial intelligence model (2206), evaluating image clarity using a second artificial intelligence model (2208), identifying anatomical regions within retained images using a third artificial intelligence model (2210), associating retained images with specific oral regions (2212), comparing retained images with historical image data to determine longitudinal condition indicators (2214), generating a condition progression alert when a longitudinal indicator exceeds a predefined threshold (2216), generating a numerical oral health score (2218), displaying longitudinal condition indicators to a user through a mobile application (2220), and transmitting anonymized image data for artificial intelligence model retraining in accordance with privacy controls (2222).

In step 2202, capturing intraoral image data using a handheld imaging device may include activating one or more illumination sources, positioning the imaging device within an oral cavity, and acquiring still images or video frames of teeth, gums, and surrounding oral structures. This step may further include providing real-time guidance to a user, such as visual prompts, overlays, or augmented-reality indicators, to assist with device positioning, focus, and coverage of target oral regions.

In step 2204, transmitting the image data to a mobile computing device may include establishing a wired or wireless communication link between the handheld imaging device and the mobile computing device and transferring the captured image data using one or more communication protocols. This step may further include compressing, encrypting, or packetizing the image data prior to transmission and confirming successful receipt of the image data by the mobile computing device.

In step 2206, filtering the image data using a first artificial intelligence model may include analyzing image content to detect non-relevant images, such as images that are out of frame, obstructed, improperly oriented, or unrelated to oral anatomy. This step may further include discarding, deprioritizing, or flagging such non-relevant images and retaining only images that satisfy predefined relevance criteria.

In step 2208, evaluating image clarity using a second artificial intelligence model may include assessing focus quality, lighting uniformity, motion blur, and image resolution to determine whether retained images meet clarity thresholds. This step may further include assigning quality scores to individual images and excluding images that fail to meet minimum clarity requirements.

In step 2210, identifying anatomical regions within retained images using a third artificial intelligence model may include detecting and segmenting oral structures such as individual teeth, gum lines, occlusal surfaces, and interproximal regions. This step may further include classifying detected anatomical regions according to predefined labels, spatial coordinates, or anatomical taxonomies.

In step 2212, associating retained images with specific oral regions may include mapping identified anatomical regions to oral region identifiers such as tooth numbers, quadrants, or gum zones. This step may further include storing metadata that links each retained image to one or more oral regions for subsequent longitudinal tracking.

In step 2214, comparing the retained images with historical image data may include retrieving prior images associated with the same oral regions and analyzing differences over time using one or more comparison metrics. This step may further include generating longitudinal condition indicators that reflect trends, progression, stability, or regression of one or more oral health conditions.

In step 2216, generating a condition progression alert may include evaluating longitudinal condition indicators against predefined thresholds indicative of increased risk or deterioration. This step may further include triggering notifications, warnings, or recommendations when one or more thresholds are exceeded.

In step 2218, generating a numerical oral health score may include aggregating survey inputs, daily hygiene inputs, and artificial intelligence-derived image analysis outputs into a composite scoring model. This step may further include weighting, normalizing, or scaling the aggregated inputs to produce a standardized oral health score.

In step 2220, displaying longitudinal condition indicators to a user through a mobile application may include rendering visual graphs, timelines, or color-coded indicators representing oral health trends. This step may further include presenting explanatory text, risk summaries, or comparative views to support user monitoring and risk awareness.

In step 2222, transmitting anonymized image data for artificial intelligence model retraining may include removing or obfuscating personally identifiable information from image data and associated metadata in accordance with privacy controls. This step may further include selectively transmitting anonymized data to a remote system for use in retraining, validating, or improving artificial intelligence models.

FIG. 23 illustrates an example user interface 2300 for guiding a device-connection workflow between a base unit 2302 and an imaging attachment 2304. In the illustrated example, the interface may present stepwise connection prompts that instruct a user to locate a connector interface and to couple the imaging attachment 2304 to the base unit 2302, and may transition to a ready state upon detecting a successful coupling event and/or communication handshake between the handheld imaging device and the mobile computing device.

In some embodiments, the mobile application provides a guided user workflow that includes onboarding interfaces, device-connection interfaces, and scan-assistance interfaces to improve usability, reduce setup errors, and increase the likelihood of acquiring diagnostically useful images. For example, upon initial execution (e.g., a first use of the application on a mobile computing device, first profile creation, or first connection of a handheld imaging device), the application may present an introductory interface that provides general informational notices and operational guidance, including non-diagnostic use notices and a user acknowledgement action that gates access to scanning functionality. The introductory interface may be displayed once per user profile, once per installation, or based on a configurable policy, and may include a selectable control that transitions the application to a device-connection workflow or scanning workflow.

In some embodiments, the application includes one or more early-access or beta interfaces that solicit user feedback and/or report known limitations. Such interfaces may include a feedback submission mechanism that allows the user to transmit application feedback, error reports, or scan-session observations. In some implementations, submitted feedback is stored together with scan-session metadata (e.g., timestamp, device model identifier, connection state, and quality metrics) to support troubleshooting and iterative improvement of scanning guidance, device connectivity logic, and image quality control.

In some embodiments, prior to enabling scanning, the application presents a “connect camera” workflow implemented as a multi-step sequence of instruction states (e.g., a carousel or stepper) that guides a user through establishing a physical and/or wireless connection between a base unit, an imaging attachment, and the mobile computing device. The workflow may include instruction states that (i) prompt a user to confirm that required hardware is present (e.g., a base unit and imaging attachment), (ii) prompt a user to locate a connector interface (e.g., a USB-C port) on the base unit or device housing, (iii) prompt a user to couple the imaging attachment to the base unit via the connector interface, and/or (iv) prompt a user to confirm readiness cues, such as a device indicator output. In some embodiments, the application permits a user to bypass one or more instruction states (e.g., via a “skip” input) while still enforcing a minimum gating requirement before scanning can begin.

In some embodiments, the handheld imaging device provides a readiness indicator output (e.g., a blinking illumination pattern or other visible indicator) representing that the imaging device has completed initialization and is ready to stream image data. The application may instruct the user to observe the readiness indicator and may additionally confirm readiness electronically by receiving a status value, handshake message, accessory enumeration signal, or streaming initialization signal from the handheld device. In some implementations, the application transitions automatically from a connection instruction state to a “ready” state when the application detects that required connection criteria have been satisfied, including one or more of: correct device identification, successful wired coupling, establishment of a Wi-Fi and/or peer-to-peer link, confirmation of sufficient battery state for scanning, and/or confirmation that an imaging attachment is present and responsive.

In some embodiments, the application enforces a connection gating rule in which image acquisition is disabled until the required connection criteria are satisfied. When the application detects a failed connection attempt (e.g., incomplete coupling, insufficient power, missing attachment, or incomplete wireless association), the application may present corrective prompts and retry logic configured to assist the user in reestablishing communication without requiring application restart. In some embodiments, initial setup uses a first communication protocol (e.g., Bluetooth) to assist pairing and configuration of a second communication protocol (e.g., Wi-Fi), and subsequent scan sessions preferentially use the second communication protocol to support higher bandwidth streaming of image data.

In some embodiments, during scanning the application provides scan-assistance interfaces that help the user acquire high-quality image data. For example, the application may evaluate scan quality in real time using one or more quality metrics, including at least one of connectivity status, illumination adequacy, estimated distance-to-target, stability/motion, or image sharpness. When one or more quality metrics fail to satisfy a predefined threshold, the application may present contextual troubleshooting guidance that identifies a likely cause and recommends corrective actions, such as confirming the mobile device network connection, verifying that the imaging attachment is fully seated, improving illumination conditions, adjusting distance between the imaging head and target anatomy, and/or moving the imaging head at a steadier speed and motion path. In some embodiments, the scan-assistance interfaces are presented dynamically during acquisition and may be integrated with the multi-layer image filtering, clarity evaluation, and anatomical association processes described herein.

In some embodiments, the mobile application presents a network-selection interface that lists available wireless networks and prompts a user to connect to a network associated with the handheld imaging device. Upon successful association, the application confirms connectivity and transitions the system to a scanning-ready state.

In some embodiments, the mobile application supports dynamic configuration and updating of artificial intelligence analysis components used for intraoral image processing. For example, the application may present an interface indicating availability or download status of one or more AI models or scan-analysis feature sets. The application may retrieve such models or feature sets from a remote system and store them locally on the mobile computing device, thereby enabling improved analysis accuracy, expanded pathology detection capabilities, or updated processing logic over time. In some implementations, the application indicates download progress and permits deferral or retry of the update process based on network availability or user preference.

In some embodiments, the mobile application provides guided scan-instruction interfaces configured to assist a user in acquiring intraoral image data from different anatomical regions. For example, the application may present region-specific scanning guidance that instructs a user on camera orientation, motion path, distance from target surfaces, and scan sequencing. Such guidance may include instructions for scanning anterior surfaces and posterior or occlusal surfaces, including recommendations to scan from one lateral side to another or from posterior regions toward anterior regions. The scan-instruction interfaces may further specify preferred distance ranges, illumination conditions, and motion stability parameters to improve image clarity and reduce motion artifacts during acquisition.

In some embodiments, the scan-instruction interfaces are presented prior to initiation of an image-acquisition session and may be selectively skipped by a user. In other embodiments, the scan-instruction interfaces are dynamically presented or updated based on detected scan progress, anatomical region identification, or quality metrics generated by the artificial intelligence analysis framework.

In some embodiments, upon completion of an image-analysis or assessment process, the mobile application presents a completion interface indicating that analysis has finished and that results are available for review. The completion interface may include a selectable control that transitions the application to an insights, findings, or results view in which analysis outputs, recommendations, or longitudinal indicators are displayed.

In some embodiments, the mobile application generates and updates a composite health or assessment score based on a combination of user-provided inputs, scan-derived findings, and artificial intelligence analysis outputs. When the composite score is updated, the application may present a notification or status interface indicating that an updated score is available and providing a control for navigating to a detailed score view or results dashboard.

In some embodiments, the mobile application coordinates the scan-instruction interfaces, assessment completion interfaces, and score-update interfaces as part of an integrated workflow that guides a user from image acquisition, through analysis, and into presentation of results. The workflow may be configured to improve scan consistency across sessions, support longitudinal tracking, and enhance user understanding of system status and available analysis outputs.

In some embodiments, the mobile application presents guided scan-instruction interfaces configured to assist a user in acquiring intraoral image data from one or more anatomical regions. The scan-instruction interfaces may provide instructions regarding camera orientation, scan sequencing, motion path, and a preferred distance range between an imaging head and target surfaces. For example, the application may instruct a user to scan anterior surfaces by moving the imaging head steadily from one lateral side to another and may further instruct scanning of additional surfaces in a defined order. The scan-instruction interfaces may be presented prior to initiation of image acquisition and may include a selectable control for proceeding to an active scanning mode.

In some embodiments, the scan-instruction interfaces are integrated with device-connection status monitoring. When the application determines that a required connection criterion has not been satisfied, the application may present a status notification indicating that image acquisition is unavailable and may prompt the user to establish or reestablish a connection with the handheld imaging device. The application may inhibit image capture while the connection criterion remains unsatisfied and may automatically enable scanning functionality once connectivity is restored.

In some embodiments, upon successful satisfaction of a connection criterion during a scan-instruction workflow, the application presents a confirmation notification indicating that the system is ready to proceed. The confirmation notification may be transient and may allow the user to continue scanning without restarting the workflow.

In some embodiments, the application presents an error notification when a connection interruption is detected during a scan-instruction or pre-scan phase. The error notification may identify a connectivity issue and provide a prompt to reconnect the handheld imaging device. The application may maintain the scan-instruction context while the connection issue is resolved, thereby allowing the user to resume scanning without repeating prior instructional steps.

In some embodiments, the mobile application provides guided scan-instruction interfaces configured to assist a user in acquiring intraoral image data from additional anatomical regions, including buccal or outer surfaces. The scan-instruction interfaces may present region-specific guidance that instructs a user on placement of an imaging head relative to target surfaces, scan sequencing, and motion direction, such as initiating acquisition near posterior regions and progressing toward anterior regions. The guidance may further specify preferred distance ranges between the imaging head and tooth surfaces, illumination conditions, and motion stability parameters to improve image clarity and reduce artifacts during acquisition.

In some embodiments, the scan-instruction interfaces for different anatomical regions are presented as part of a multi-step instructional sequence and may include an option for a user to skip one or more instruction steps. In other embodiments, the scan-instruction interfaces are dynamically coordinated with detected scan progress or anatomical region identification performed by the artificial intelligence analysis framework.

In some embodiments, the mobile application presents one or more legal or privacy notice interfaces during initial use, account creation, or prior to enabling scanning or analysis functionality. Such interfaces may present privacy policies, data-use disclosures, and jurisdiction-specific notices, and may require an affirmative user acknowledgment prior to continued operation of the application. The notices may address, by way of example, collection, use, storage, and sharing of personal information, user rights under applicable privacy regulations, and procedures for exercising such rights.

In some embodiments, the mobile application presents health-information privacy notices applicable to handling of medical or dental information. The application may require user acknowledgment of such notices before enabling features that involve storage, analysis, or transmission of scan data or related health information. Acceptance or rejection of the notices may control whether certain features are enabled, limited, or disabled.

In some embodiments, the application stores acknowledgment status of presented legal or privacy notices in association with a user profile and may suppress repeated presentation of such notices once accepted, unless an updated notice is provided or required by a change in applicable regulations or system configuration.

In some embodiments, the mobile application provides an authentication workflow that enables a user to securely access application functionality. The authentication workflow may include entry of credential information and verification prior to allowing access to scanning, analysis, or profile-associated features. Upon successful authentication, the application associates subsequent scan sessions, analysis outputs, and longitudinal tracking data with a corresponding user profile.

In some embodiments, the mobile application presents one or more legal review interfaces that require a user to review and affirmatively accept applicable legal documentation prior to continued use of the application. Such documentation may include terms of use, privacy policies, and health-information privacy notices. Acceptance or rejection of the legal documentation may control whether scanning, data storage, analysis, or data transmission features are enabled or restricted.

In some embodiments, the mobile application detects when required prerequisite information for generating an assessment output is unavailable. For example, when a composite health or assessment score cannot be generated due to missing user inputs or incomplete onboarding information, the application may present a notification prompting completion of a survey, questionnaire in some embodiments, the mobile application presents an active scanning interface during acquisition of intraoral image data. The active scanning interface may visually indicate scan progress relative to one or more predefined oral regions, such as left and right sides or upper and lower regions, and may identify a current target region being scanned. The interface may update dynamically as image data is received, thereby providing real-time feedback regarding scan coverage and acquisition status.

In some embodiments, the active scanning interface includes one or more scan-control inputs that allow a user to temporarily pause or resume image acquisition. When scanning is paused, the application may suspend capture or streaming of image data while maintaining scan-progress context, thereby enabling the user to resume acquisition without restarting the scan sequence.

In some embodiments, the active scanning interface may display a live image preview or representative frame corresponding to a region currently being scanned. The live preview may be used by the application to evaluate image quality metrics, guide scan progression, and determine when sufficient image data has been collected for a particular oral region.

In some embodiments, upon completion of a scan and associated analysis, the mobile application presents an assessment-completion interface indicating that image processing has finished and that analysis results are available. The assessment-completion interface may include a selectable control that transitions the application to an insights or results view in which findings, recommendations, or longitudinal indicators are displayed.

In some embodiments, the mobile application presents a launch or splash interface during application startup. The launch interface may be displayed while application resources are initialized, connections are established, or configuration data is loaded, and may transition automatically to an authentication, onboarding, or scanning workflow once initialization is complete.re, or other required input prior to updating or displaying the score.

In some embodiments, the mobile application presents an initial assessment or welcome survey interface configured to collect baseline user information relevant to oral health monitoring and personalization. The welcome survey may include a sequence of questions presented one at a time and may accept user responses through selectable options. Responses may be stored in association with a user profile and used to initialize or influence analysis parameters, recommendation logic, reminder behavior, or composite health or engagement metrics.

In some embodiments, the application enforces completion requirements for the welcome survey. When required questions are skipped or omitted, the application may present a notification indicating that additional input is needed to complete the assessment. The notification may prompt the user to return to unanswered questions prior to submission of the survey.

In some embodiments, the application provides a controlled skip or exit workflow for the welcome survey. For example, when a user attempts to exit the assessment before completion, the application may present an option to continue the assessment or to save partial progress and exit. Saved progress may be automatically restored when the user later resumes the assessment, thereby reducing user friction while preserving data continuity.

In some embodiments, the mobile application presents an active scanning interface that includes a timing indicator associated with acquisition of image data for a particular oral region. The timing indicator may represent a remaining or elapsed acquisition interval and may be coordinated with region-based scan guidance to ensure sufficient image coverage is collected for each region prior to advancing to a subsequent region.

In some embodiments, the timing indicator, scan-progress representation, and assessment workflows operate together as part of an integrated system that guides a user through data collection and assessment completion. The integrated system may be configured to improve consistency of data acquisition, enforce minimum data-collection thresholds, and support longitudinal comparison of scan results across multiple sessions.

In some embodiments, prior to presenting individual questions of an initial assessment or welcome survey, the mobile application presents an introductory assessment interface that explains the purpose of the survey and its role in improving personalization, accuracy of analysis outputs, and reminder behavior. The introductory interface may inform a user that completion of the survey improves assessment accuracy and may further indicate that skipping the survey may reduce personalization or confidence of generated metrics.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

Performance characteristics described herein, including time durations, ranges, and capacity values, are illustrative and may vary based on implementation.

What is claimed is:

1. A health monitoring system, comprising:
a handheld intraoral imaging device including an image sensor, a lens assembly, illumination sources, a wireless communication module, a processor, and memory;
a mobile computing device executing a mobile application; and
a multi-layer artificial intelligence (AI) analysis framework executed by at least one of the handheld device and the mobile computing device,
wherein the handheld intraoral imaging device is configured to capture intraoral image data and transmit the image data to the mobile computing device, and
wherein the multi-layer AI analysis framework is configured to:
filter non-relevant or low-quality images,
identify anatomical regions within retained images,
associate retained images with specific teeth or oral regions, and
generate longitudinal condition indicators based on comparison of historical and current image data,
and wherein the mobile application presents the longitudinal condition indicators to a user for monitoring and risk-awareness purposes.

2. The system of claim 1, wherein the multi-layer AI analysis framework includes a classifier model, a clarity evaluation model, and a tooth identification model.

3. The system of claim 1, wherein the handheld intraoral imaging device includes an active lens heating element configured to reduce condensation on the lens.

4. The system of claim 1, wherein the illumination sources comprise a plurality of light emitting diodes positioned circumferentially around the lens assembly.

5. The system of claim 1, wherein the handheld device includes a modular coupling interface configured to receive interchangeable imaging heads.

6. The system of claim 5, wherein at least one interchangeable imaging head comprises a fluorescence-based imaging head.

7. The system of claim 5, wherein at least one interchangeable imaging head comprises a hyperspectral imaging head.

8. The system of claim 1, wherein the mobile application provides augmented-reality guidance overlays during image capture.

9. The system of claim 1, wherein the mobile application generates a composite oral health score based on survey data, daily behavior inputs, and AI-derived image analysis outputs.

10. The system of claim 1, wherein the mobile application maintains a longitudinal condition monitoring feature configured to alert a user when a tracked condition exceeds a predefined change threshold.

11. A method of monitoring oral health conditions, comprising:
capturing intraoral image data using a handheld imaging device;
transmitting the image data to a mobile computing device;
filtering the image data using a first artificial intelligence model to remove non-relevant images;
evaluating image clarity using a second artificial intelligence model;
identifying anatomical regions within retained images using a third artificial intelligence model;
associating the retained images with specific oral regions;
comparing the retained images with historical image data to determine longitudinal condition indicators; and
displaying the longitudinal condition indicators to a user through a mobile application for monitoring and risk-awareness purposes.

12. The method of claim 11, further comprising generating a condition progression alert when a longitudinal indicator exceeds a predefined threshold.

13. The method of claim 11, further comprising generating a numerical oral health score based on survey inputs, daily hygiene inputs, and AI-derived image analysis outputs.

14. The method of claim 11, further comprising providing augmented-reality guidance during image capture.

15. The method of claim 11, further comprising transmitting anonymized image data for AI model retraining in accordance with privacy controls.
